(19) United States
(12) Patent Application Publication
(10) Pub. No.: US 2011/0121269 A1
(43) Pub. Date: May 26, 2011

(54) DEUTERATED COMPOUNDS FOR ELECTRONIC APPLICATIONS

(75) Inventors: DANIEL DAVID LECLOUX, WILMINGTON, DE (US); ADAM FENNIMORE, WILMINGTON, DE (US); WEIYING GAO, LANDENBERG, PA (US); NORA SABINA RADU, LANDENBERG, PA (US); WEISHI WU, LANDENBERG, PA (US); VSEVOLOD ROSTOVTSEV, SWARTHMORE, PA (US); MICHAEL HENRY HOWARD, JR., MONTCHANIN, DE (US); HONG MENG, WILMINGTON, DE (US); YULONG SHEN, WILMINGTON, DE (US)

(73) Assignee: E.I. DU PONT DE NEMOURS AND COMPANY, Wilmington, DE (US)

(21) Appl. No.: 12/782,781

(22) Filed: May 19, 2010

Related U.S. Application Data
(60) Provisional application No. 61/179,407, filed on May 19, 2009.

(51) Int. Cl. H01L 51/54; C07C 211/54; C07C 25/22; C07C 25/02; C07F 5/02; C07C 13/573

(52) U.S. Cl. 257/40; 564/426; 570/183; 570/182; 568/6; 585/26; 257/E51.041

(57) ABSTRACT

This invention relates to deuterated aryl-anthracene compounds that are useful in electronic applications. It also relates to electronic devices in which the active layer includes such a deuterated compound.

### RELATED APPLICATION

This application claims priority under 35 U.S.C. §119(e) from Provisional Application No. 61/179,407 filed May 19, 2009 which is incorporated by reference in its entirety.

### BACKGROUND

1. Field of the Disclosure

This invention relates to anthracene derivative compounds which are at least partially deuterated. It also relates to electronic devices in which the active layers include such a compound.

2. Description of the Related Art

Organic electronic devices that emit light, such as light-emitting diodes that make up displays, are present in many different kinds of electronic equipment. In all such devices, an organic active layer is sandwiched between two electrical contact layers. At least one of the electrical contact layers is light-transmitting so that light can pass through the electrical contact layer. The organic active layer emits light through the light-transmitting electrical contact layer upon application of electricity across the electrical contact layers.

It is well known to use organic electroluminescent compounds as the active component in light-emitting diodes. Simple organic molecules such as anthracene, thiadiazole derivatives, and coumarin derivatives are known to show electroluminescence. Semiconductive conjugated polymers have also been used as electroluminescent components, as has been disclosed in, for example, U.S. Pat. No. 5,247,190, U.S. Pat. No. 5,408,109, and Published European Patent Application 443 861.

In many cases the electroluminescent compound is present in a host material. There is a continuing need for new host compounds.

### SUMMARY

There is provided an aryl-substituted anthracene having at least one deuterium element D.

There is also provided an electronic device comprising an active layer comprising the above compound.

### BRIEF DESCRIPTION OF THE DRAWINGS

Embodiments are illustrated in the accompanying figures to improve understanding of concepts as presented herein.

FIG. 1 includes an illustration of one example of an organic electronic device.

FIG. 2 includes the ¹H NMR spectrum of the comparative compound of Comparative Example A.

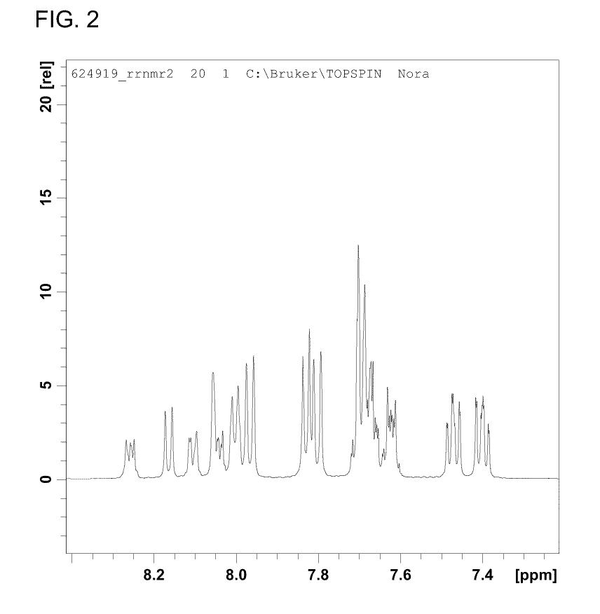

FIG. 3 includes the ¹H NMR spectrum of the deuterated compound of Example 1.

FIG. 4 includes the mass spectrum of the deuterated compound of Example 1.

Skilled artisans appreciate that objects in the figures are illustrated for simplicity and clarity and have not necessarily been drawn to scale.

For example, the dimensions of some of the objects in the figures may be exaggerated relative to other objects to help to improve understanding of embodiments.

### DETAILED DESCRIPTION

Many aspects and embodiments are disclosed herein and are exemplary and not limiting. After reading this specification, skilled artisans appreciate that other aspects and embodiments are possible without departing from the scope of the invention.

Other features and benefits of any one or more of the embodiments will be apparent from the following detailed description, and from the claims. The detailed description first addresses Definitions and Clarification of Terms followed by the Deuterated Compound, the Electronic Device, and finally Examples.

### 1. DEFINITIONS AND CLARIFICATION OF TERMS

Before addressing details of embodiments described below, some terms are defined or clarified.

As used herein, the term “aliphatic ring” is intended to mean a cyclic group that does not have delocalized pi electrons. In some embodiments, the aliphatic ring has no unsaturation. In some embodiments, the ring has one double or triple bond.

The term “alkoxy” refers to the group RO—, where R is an alkyl.

The term “alkyl” is intended to mean a group derived from an aliphatic hydrocarbon having one point of attachment, and includes a linear, a branched, or a cyclic group. The term is intended to include heteroalkyls. The term “hydrocarbon alkyl” refers to an alkyl group having no heteroatoms. The term “deuterated alkyl” is a hydrocarbon alkyl having at least one available H replaced by D. In some embodiments, an alkyl group has from 1-20 carbon atoms.

The term “branched alkyl” refers to an alkyl group having at least one secondary or tertiary carbon. The term “secondary alkyl” refers to a branched alkyl group having a secondary carbon atom. The term “tertiary alkyl” refers to a branched alkyl group having a tertiary carbon atom. In some embodiments, the branched alkyl group is attached via a secondary or tertiary carbon.

The term “aryl” is intended to mean a group derived from an aromatic hydrocarbon having one point of attachment. The term “aromatic compound” is intended to mean an organic compound comprising at least one unsaturated cyclic group having delocalized pi electrons. The term is intended include heteroaryls. The term “hydrocarbon aryl” is intended to mean aromatic compounds having no heteroatoms in the ring. The term aryl includes groups which have a single ring and those which have multiple rings which can be joined by a single bond or fused together. The term “deuterated aryl” refers to an aryl group having at least one available H bonded directly to the aryl replaced by D. The term “arylene” is intended to mean a group derived from an aromatic hydrocarbon having two points of attachment. In some embodiments, an aryl group has from 3-60 carbon atoms.

The term “aryloxy” refers to the group RO—, where R is an aryl.

The term “compound” is intended to mean an electrically uncharged substance made up of molecules that further consist of atoms, wherein the atoms cannot be separated by physical means. The phrase “adjacent to,” when used to refer to layers in a device, does not necessarily mean that one layer is immediately next to another layer. On the other hand, the phrase “adjacent R groups,” is used to refer to R groups that are next to each other in a chemical formula (i.e., R groups that are on atoms joined by a bond). The term “photoactive” refers to any material that exhibits electroluminescence and/or photosensitivity.

The term “deuterated” is intended to mean that at least one H has been replaced by D. The deuterium is present in at least 100 times the natural abundance level.

The prefix “hetero” indicates that one or more carbon atoms have been replaced with a different atom. In some embodiments, the different atom is N, O, or S.

The term “layer” is used interchangeably with the term “film” and refers to a coating covering a desired area. The term is not limited by size. The area can be as large as an entire device or as small as a specific functional area such as the actual visual display, or as small as a single sub-pixel. Layers and films can be formed by any conventional deposition technique, including vapor deposition, liquid deposition (continuous and discontinuous techniques), and thermal transfer. Continuous deposition techniques, include but are not limited to, spin coating, gravure coating, curtain coating, dip coating, slot-die coating, spray coating, and continuous nozzle coating. Discontinuous deposition techniques include, but are not limited to, ink jet printing, gravure printing, and screen printing.

The term “organic electronic device” or sometimes just “electronic device” is intended to mean a device including one or more organic semiconductor layers or materials. All groups can be substituted or unsubstituted unless otherwise indicated. In some embodiments, the substituents are selected from the group consisting of D, halide, alkyl, alkoxy, aryl, aryloxy, cyano, and NR₂, where R is alkyl or aryl.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The IUPAC numbering system is used throughout, where the groups from the Periodic Table are numbered from left to right as 1-18 (CRC Handbook of Chemistry and Physics, 81st Edition, 2000).

### 2. DEUTERATED COMPOUND

The new deuterated compound is an aryl-substituted anthracene compound having at least one D. In some embodiments, the compound is at least 10% deuterated. By this is meant that at least 10% of the H are replaced by D. In some embodiments, the compound is at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated. In some embodiments, the compounds are 100% deuterated.

In one embodiment, the deuterated compound has Formula I:

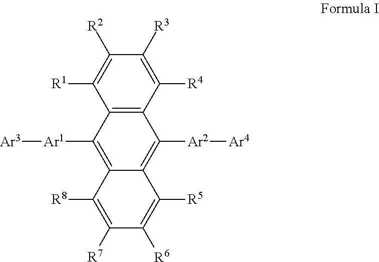

wherein:
R¹ through R⁸ are the same or different at each occurrence and are selected from the group consisting of H, D, alkyl, alkoxy, aryl, aryloxy, diarylamino, siloxane, and silyl;
Ar¹ and Ar² are the same or different and are selected from the group consisting of aryl groups; and
Ar³ and Ar⁴ are the same or different and are selected from the group consisting of H, D, and aryl groups;
   wherein the compound has at least one D.

In some embodiments of Formula I, the at least one D is on a substituent group on an aryl ring. In some embodiments, the substituent group is selected from alkyl, aryl, and diarylamino.

In some embodiments of Formula I, at least one of R¹ through R⁸ is D. In some embodiments, at least two of R¹ through R⁸ are D. In some embodiments, at least three are D; in some embodiments, at least four are D; in some embodiments, at least five are D; in some embodiments, at least six are D; in some embodiments, at least seven are D. In some embodiments, all of R¹ through R⁸ are D.

In some embodiments, R¹ through R⁸ are selected from H and D. In some embodiments, one of R¹ through R⁸ are D and seven are H. In some embodiments, two of R¹ through R⁸ are D and six are H. In some embodiments, three of R¹ through R⁸ are D and five are H. In some embodiments, four of R¹ through R⁸ are D, and four are H. In some embodiments, five of R¹ through R⁸ are D and three are H. In some embodiments, six of R¹ through R⁸ are D and two are H. In some embodiments, seven of R¹ through R⁸ are D and one is H. In some embodiments, eight of R¹ through R⁸ are D.

In some embodiments, at least one of R¹ through R⁸ is selected from alkyl, alkoxy, aryl, aryloxy, diarylamino, siloxane, and silyl, and the remainder of R¹ through R⁸ are selected from H and D. In some embodiments, R² is selected from alkyl, alkoxy, aryl, aryloxy, diarylamino, siloxane, and silyl. In some embodiments, R² is selected from alkyl and aryl. In some embodiments, R² is selected from deuterated alkyl and deuterated aryl. In some embodiments, R² is selected from deuterated aryl having at least 10% deuteration. In some embodiments, R² is selected from deuterated aryl having at least 20% deuteration; in some embodiments, at least 30% deuteration; in some embodiments, at least 40% deuteration; in some embodiments, at least 50% deuteration; in some embodiments, at least 60% deuteration; in some embodiments, at least 70% deuteration; in some embodiments, at least 80% deuteration; in some embodiments, at least 90% deuteration. In some embodiments, R² is selected from deuterated aryl having 100% deuteration.

In some embodiments of Formula I, at least one of Ar¹ through Ar⁴ is a deuterated aryl. In some embodiments, Ar³ and Ar⁴ are selected from D and deuterated aryls.

In some embodiments of Formula I, Ar¹ through Ar⁴ are at least 10% deuterated. In some embodiments of Formula I, Ar¹ through Ar⁴ are at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated; in some embodiments, 100% deuterated.

In some embodiments, the compound of Formula I is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated. In some embodiments, the compound is 100% deuterated.

In some embodiments, Ar¹ and Ar² are selected from the group consisting of phenyl, naphthyl, phenanthryl, and anthracenyl. In some embodiments, Ar¹ and Ar² are selected from the group consisting of phenyl and naphthyl.

In some embodiments, Ar³ and Ar⁴ are selected from the group consisting of phenyl, naphthyl, phenanthryl, anthracenyl, phenylnaphthylene, naphthylphenylene, and a group having Formula II:

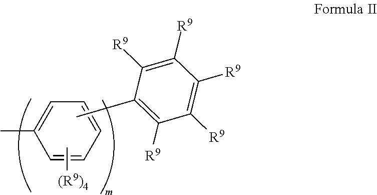

where:
R⁹ is the same or different at each occurrence and is selected from the group consisting of H, D, alkyl, alkoxy, diarylamino, siloxane and silyl, or adjacent R⁹ groups may be joined together to form an aromatic ring; and
m is the same or different at each occurrence and is an integer from 1 to 6.

In some embodiments, Ar³ and Ar⁴ are selected from the group consisting of phenyl, naphthyl, phenylnaphthylene, naphthylphenylene, and a group having Formula III:

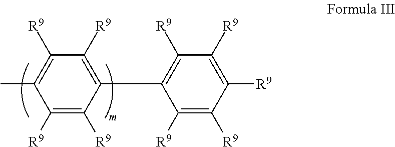

where R⁹ and m are as defined above for Formula II. In some embodiments, m is an integer from 1 to 3.

In some embodiments, at least one of Ar¹ through Ar⁴ is a heteroaryl group. In some embodiments, the heteroaryl group is selected from carbazole, benzofuran, and dibenzofuran. In some embodiments, the heteroaryl group is deuterated. In some embodiments, the heteroaryl group is at least 10% deuterated; in some embodiments, at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated. In some embodiments, the heteroaryl group is 100% deuterated.

In some embodiments of Formula I, at least one of R¹ through R⁸ is D and at least one of Ar¹ through Ar⁴ is a deuterated aryl. In some embodiments, the compound is at least 10% deuterated. In some embodiments, the compound is at least 20% deuterated; in some embodiments, at least 30% deuterated; in some embodiments, at least 40% deuterated; in some embodiments, at least 50% deuterated; in some embodiments, at least 60% deuterated; in some embodiments, at least 70% deuterated; in some embodiments, at least 80% deuterated; in some embodiments, at least 90% deuterated. In some embodiments, the compound is 100% deuterated.

Some non-limiting examples of compounds having Formula I include Compounds H1 through H13 below:

### Compound H1:

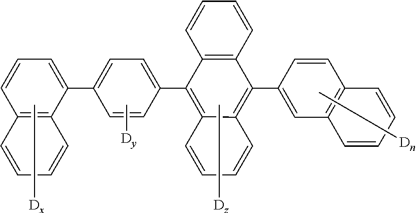 where x+y+z+n=1-26

### Compound H2:

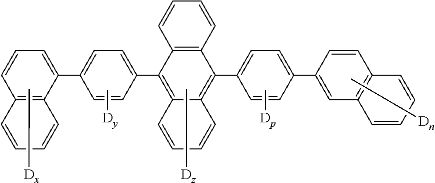 where x+y+z+p+n=1-30

### Compound H3:

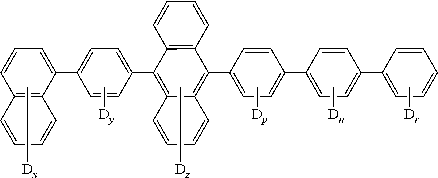 where x+y+z+p+n+r=1-32

### Compound H4:

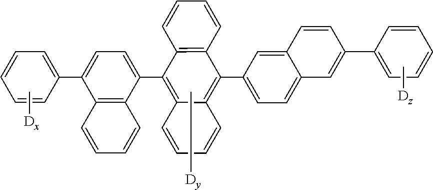 where x+y+z+p+n=1-18

### Compound H5:

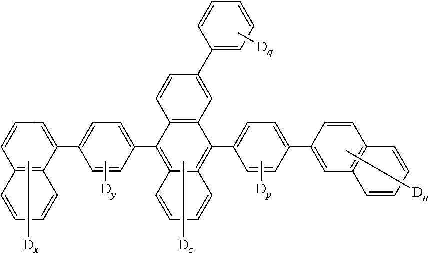 where x+y+z+p+n+q=1-34

### Compound H6:

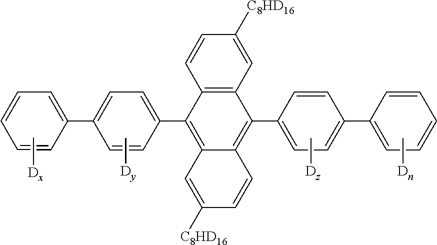 where x+y+z+n=1-18

### Compound H7:

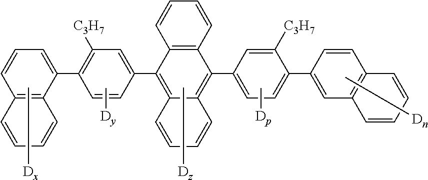 where x+y+z+p+n=1-28

### Compound H8:

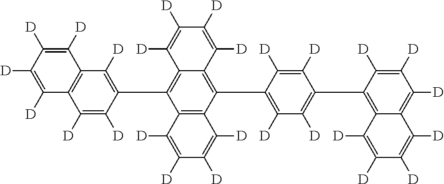

### Compound H9:

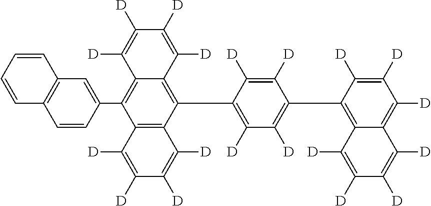

### Compound H10:

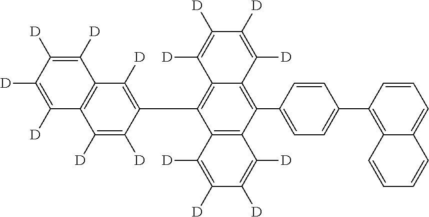

### Compound H11:

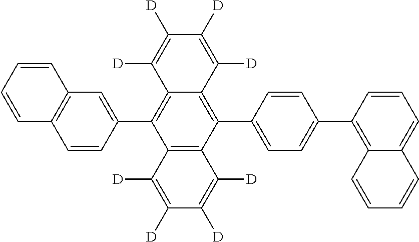

### Compound H12:

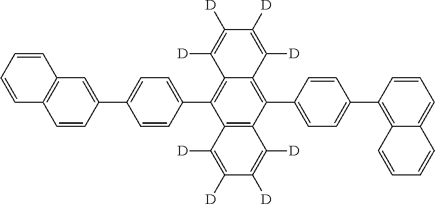

### Compound H13:

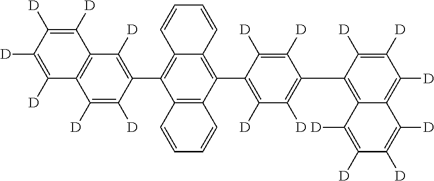

The non-deuterated analogs of the new compounds can be prepared by known coupling and substitution reactions. The new deuterated compound can then be prepared in a similar manner using deuterated precursor materials or, more generally, by treating the non-deuterated compound with deuterated solvent, such as d6-benzene, in the presence of a Lewis acid H/D exchange catalyst, such as aluminum trichloride or ethyl aluminum chloride, or acids such as CF₃COOD, DCl, etc. Exemplary preparations are given in the Examples. The level of deuteration can be determined by NMR analysis and by mass spectrometry, such as Atmospheric Solids Analysis Probe Mass Spectrometry (ASAP-MS). The starting materials of the perdeuterated or partially deuterated aromatic compounds or alky compounds can be purchased from the commercial source or can be obtained using known methods. Some examples of such methods can be found in a) “Efficient H/D Exchange Reactions of Alkyl-Substituted Benzene Derivatives by Means of the Pd/C—H2-D2O System” Hiroyoshi Esaki, Fumiyo Aoki, Miho Umemura, Masatsugu Kato, Tomohiro Maegawa, Yasunari Monguchi, and Hironao Sajiki Chem. Eur. J. 2007, 13, 4052-4063. b) “Aromatic H/D Exchange Reaction Catalyzed by Groups 5 and 6 Metal Chlorides” GUO, Qiao-Xia, SHEN, Bao-Jian; GUO, Hai-Qing TAKAHASHI, Tamotsu Chinese Journal of Chemistry, 2005, 23, 341-344; c) “A novel deuterium effect on dual charge-transfer and ligand-field emission of the cis-dichlorobis(2,2′-bipyridine)iridium(III) ion” Richard J. Watts, Shlomo Efrima, and Horia Metiu J. Am. Chem. Soc., 1979, 101 (10), 2742-2743; d) “Efficient H-D Exchange of Aromatic Compounds in Near-Critical D20 Catalysed by a Polymer-Supported Sulphonic Acid” Carmen Boix and Martyn Poliakoff Tetrahedron Letters 40 (1999) 4433-4436; e) U.S. Pat. No. 3,849,458; f) “Efficient C—H/C-D Exchange Reaction on the Alkyl Side Chain of Aromatic Compounds Using Heterogeneous Pd/C in D20” Hironao Sajiki, Fumiyo Aoki, Hiroyoshi Esaki, Tomohiro Maegawa, and Kosaku Hirota Org. Lett., 2004, 6 (9), 1485-1487.

The compounds described herein can be formed into films using liquid deposition techniques. Surprisingly and unexpectedly, these compounds have greatly improved properties when compared to analogous non-deuterated compounds. Electronic devices including an active layer with the compounds described herein, have greatly improved lifetimes. In addition, the lifetime increases are achieved in combination with high quantum efficiency and good color saturation. Furthermore, the deuterated compounds described herein have greater air tolerance than the non-deuterated analogs. This can result in greater processing tolerance both for the preparation and purification of the materials and in the formation of electronic devices using the materials.

### 3. ELECTRONIC DEVICE

Organic electronic devices that may benefit from having one or more layers comprising the electroluminescent materials described herein include, but are not limited to, (1) devices that convert electrical energy into radiation (e.g., a light-emitting diode, light emitting diode display, or diode laser), (2) devices that detect signals through electronics processes (e.g., photodetectors, photoconductive cells, photoresistors, photoswitches, phototransistors, phototubes, IR detectors), (3) devices that convert radiation into electrical energy, (e.g., a photovoltaic device or solar cell), and (4) devices that include one or more electronic components that include one or more organic semi-conductor layers (e.g., a transistor or diode).

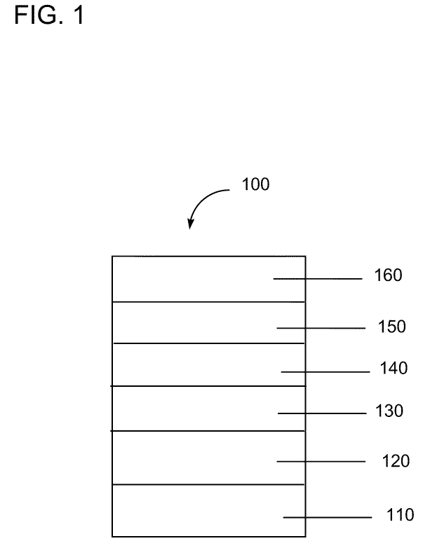

One illustration of an organic electronic device structure is shown in FIG. 1. The device 100 has a first electrical contact layer, an anode layer 110 and a second electrical contact layer, a cathode layer 160, and a photoactive layer 140 between them. Adjacent to the anode is a buffer layer 120. Adjacent to the buffer layer is a hole transport layer 130, comprising hole transport material. Adjacent to the cathode may be an electron transport layer 150, comprising an electron transport material. As an option, devices may use one or more additional hole injection or hole transport layers (not shown) next to the anode 110 and/or one or more additional electron injection or electron transport layers (not shown) next to the cathode 160.

Layers 120 through 150 are individually and collectively referred to as the active layers.

In one embodiment, the different layers have the following range of thicknesses: anode 110, 500-5000 Å, in one embodiment 1000-2000 Å; buffer layer 120, 50-2000 Å, in one embodiment 200-1000 Å; hole transport layer 130, 50-2000 Å, in one embodiment 200-1000 Å; photoactive layer 140, 10-2000 Å, in one embodiment 100-1000 Å; layer 150, 50-2000 Å, in one embodiment 100-1000 Å; cathode 160, 200-10000 Å, in one embodiment 300-5000 Å. The location of the electron-hole recombination zone in the device, and thus the emission spectrum of the device, can be affected by the relative thickness of each layer. The desired ratio of layer thicknesses will depend on the exact nature of the materials used.

Depending upon the application of the device 100, the photoactive layer 140 can be a light-emitting layer that is activated by an applied voltage (such as in a light-emitting diode or light-emitting electrochemical cell), or a layer of material that responds to radiant energy and generates a signal with or without an applied bias voltage (such as in a photodetector). Examples of photodetectors include photoconductive cells, photoresistors, photoswitches, phototransistors, and phototubes, and photovoltaic cells, as these terms are described in Markus, John, Electronics and Nucleonics Dictionary, 470 and 476 (McGraw-Hill, Inc. 1966).

One or more of the new deuterated materials described herein may be present in one or more of the active layers of a device. The deuterated materials may be used alone or in combination with non-deuterated materials.

In some embodiments, the new deuterated compounds are useful as hole transport materials in layer 130. In some embodiments, at least one additional layer includes a new deuterated material. In some embodiments, the additional layer is the buffer layer 120. In some embodiments, the additional layer is the photoactive layer 140. In some embodiments, the additional layer is the electron transport layer 150.

In some embodiments, the new deuterated compounds are useful as host materials for photoactive materials in photoactive layer 140. In some embodiments, the emissive material is also deuterated. In some embodiments, at least one additional layer includes a deuterated material. In some embodiments, the additional layer is the buffer layer 120. In some embodiments, the additional layer is the hole transport layer 130. In some embodiments, the additional layer is the electron transport layer 150

In some embodiments, the new deuterated compounds are useful as electron transport materials in layer 150. In some embodiments, at least one additional layer includes a deuterated material. In some embodiments, the additional layer is the buffer layer 120. In some embodiments, the additional layer is the hole transport layer 130. In some embodiments, the additional layer is the photoactive layer 140.

In some embodiments, an electronic device has deuterated materials in any combination of layers selected from the group consisting of the buffer layer, the hole transport layer, the photoactive layer, and the electron transport layer.

In some embodiments, the devices have additional layers to aid in processing or to improve functionality. Any or all of these layers can include deuterated materials. In some embodiments, all the organic device layers comprise deuterated materials. In some embodiments, all the organic device layers consist essentially of deuterated materials.

a. Photoactive Layer

The new deuterated compounds of Formula I are useful as hosts for photoactive materials in layer 140. The compounds can be used alone, or in combination with a second host material. The new deuterated compounds can be used as a host for materials with any color of emission. In some embodiments, the new deuterated compounds are used as hosts for green- or blue-emissive materials.

In some embodiments, the photoactive layer consists essentially of a host material having Formula I and one or more electroluminescent compounds.

In some embodiments, the new deuterated compound described herein is an electroluminescent material and is present as a photoactive material. Other EL materials which can be used in the devices, include, but are not limited to, small molecule organic fluorescent compounds, fluorescent and phosphorescent metal complexes, conjugated polymers, and mixtures thereof. Examples of fluorescent compounds include, but are not limited to, chrysenes, pyrenes, perylenes, rubrenes, coumarins, anthracenes, thiadiazoles, derivatives thereof, and mixtures thereof. Examples of metal complexes include, but are not limited to, metal chelated oxinoid compounds, such as tris(8-hydroxyquinolato)aluminum (Alq3); cyclometalated iridium and platinum electroluminescent compounds, such as complexes of iridium with phenylpyridine, phenylquinoline, or phenylpyrimidine ligands as disclosed in Petrov et al., U.S. Pat. No. 6,670,645 and Published PCT Applications WO 03/063555 and WO 2004/016710, and organometallic complexes described in, for example, Published PCT Applications WO 03/008424, WO 03/091688, and WO 03/040257, and mixtures thereof. Examples of conjugated polymers include, but are not limited to poly(phenylenevinylenes), polyfluorenes, poly(spirobifluorenes), polythiophenes, poly(p-phenylenes), copolymers thereof, and mixtures thereof.

In some embodiments, the photoactive dopant is a cyclometalated complex of iridium. In some embodiments, the complex has two ligands selected from phenylpyridines, phenylquinolines, and phenylisoquinolines, and a third liqand with is a β-dienolate. The ligands may be unsubstituted or substituted with F, D, alkyl, CN, or aryl groups.

In some embodiments, the photoactive dopant is a polymer selected from the group consisting of poly(phenylenevinylenes), polyfluorenes, and polyspirobifluorenes.

In some embodiments, the photoactive dopant is selected from the group consisting of a non-polymeric spirobifluorene compound and a fluoranthene compound.

In some embodiments, the photoactive dopant is a compound having aryl amine groups. In some embodiments, the photoactive dopant is selected from the formulae below:

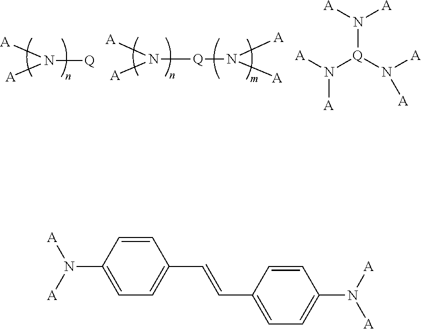

where:

A is the same or different at each occurrence and is an aromatic group having from 3-60 carbon atoms;

Q is a single bond or an aromatic group having from 3-60 carbon atoms;

n and m are independently an integer from 1-6.

In some embodiments of the above formula, at least one of A and Q in each formula has at least three condensed rings. In some embodiments, m and n are equal to 1.

In some embodiments, Q is a styryl or styrylphenyl group.

In some embodiments, Q is an aromatic group having at least two condensed rings. In some embodiments, Q is selected from the group consisting of naphthalene, anthracene, chrysene, pyrene, tetracene, xanthene, perylene, coumarin, rhodamine, quinacridone, and rubrene.

In some embodiments, A is selected from the group consisting of phenyl, tolyl, naphthyl, and anthracenyl groups.

In some embodiments, the photoactive dopant has the formula below:

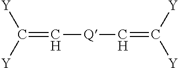

where:

Y is the same or different at each occurrence and is an aromatic group having 3-60 carbon atoms;

Q′ is an aromatic group, a divalent triphenylamine residue group, or a single bond.

In some embodiments, the photoactive dopant is an aryl acene. In some embodiments, the photoactive dopant is a non-symmetrical aryl acene.

In some embodiments, the photoactive dopant is a chrysene derivative. The term “chrysene” is intended to mean 1,2-benzophenanthrene. In some embodiments, the photoactive dopant is a chrysene having aryl substituents. In some embodiments, the photoactive dopant is a chrysene having arylamino substituents. In some embodiments, the photoactive dopant is a chrysene having two different arylamino substituents. In some embodiments, the chrysene derivative has a deep blue emission.

In some embodiments, the photoactive dopant is selected from the group consisting of amino-substituted chrysenes and amino-substituted anthracenes.

b. Other Device Layers

The other layers in the device can be made of any materials that are known to be useful in such layers.

The anode 110, is an electrode that is particularly efficient for injecting positive charge carriers. It can be made of, for example, materials containing a metal, mixed metal, alloy, metal oxide or mixed-metal oxide, or it can be a conducting polymer, or mixtures thereof. Suitable metals include the Group 11 metals, the metals in Groups 4-6, and the Group 8-10 transition metals. If the anode is to be light-transmitting, mixed-metal oxides of Groups 12, 13 and 14 metals, such as indium-tin-oxide, are generally used. The anode 110 can also comprise an organic material such as polyaniline as described in “Flexible light-emitting diodes made from soluble conducting polymer,” Nature vol. 357, pp 477-479 (11 Jun. 1992). At least one of the anode and cathode is desirably at least partially transparent to allow the generated light to be observed.

The buffer layer 120 comprises buffer material and may have one or more functions in an organic electronic device, including but not limited to, planarization of the underlying layer, charge transport and/or charge injection properties, scavenging of impurities such as oxygen or metal ions, and other aspects to facilitate or to improve the performance of the organic electronic device. Buffer materials may be polymers, oligomers, or small molecules. They may be vapour deposited or deposited from liquids which may be in the form of solutions, dispersions, suspensions, emulsions, colloidal mixtures, or other compositions.

The buffer layer can be formed with polymeric materials, such as polyaniline (PANI) or polyethylenedioxythiophene (PEDOT), which are often doped with protonic acids. The protonic acids can be, for example, poly(styrenesulfonic acid), poly(2-acrylamido-2-methyl-1-propanesulfonic acid), and the like.

The buffer layer can comprise charge transfer compounds, and the like, such as copper phthalocyanine and the tetrathiafulvalene-tetracyanoquinodimethane system (TTF-TCNQ).

In some embodiments, the buffer layer comprises at least one electrically conductive polymer and at least one fluorinated acid polymer. Such materials have been described in, for example, published U.S. patent applications 2004-0102577, 2004-0127637, and 2005/205860

In some embodiments, the hole transport layer 130 comprises the new deuterated compound of Formula I. Examples of other hole transport materials for layer 130 have been summarized for example, in Kirk-Othmer Encyclopedia of Chemical Technology, Fourth Edition, Vol. 18, p. 837-860, 1996, by Y. Wang. Both hole transporting molecules and polymers can be used. Commonly used hole transporting molecules are: N,N′-diphenyl-N,N′-bis(3-methylphenyl)-[1,1-biphenyl]-4,4′-diamine (TPD), 1,1-bis[(di-4-tolylamino) phenyl]cyclohexane (TAPC), N,N′-bis(4-methylphenyl)-N,N′-bis(4-ethylphenyl)-[1,1-(3,3′-dimethyl)biphenyl]-4,4′-diamine (ETPD), tetrakis-(3-methylphenyl)-N,N,N′,N′-2,5-phenylenediamine (PDA), a-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]-5-[p-(diethylamino)phenyl]pyrazoline (PPR or DEASP), 1,2-trans-bis(9H-carbazol-9-yl)cyclobutane (DCZB), N,N,N′,N′-tetrakis(4-methylphenyl)-(1,1-biphenyl)-4,4′-diamine (TTB), N,N′-bis(naphthalen-1-yl)-N,N′-bis-(phenyl)benzidine (—NPB), and porphyrinic compounds, such as copper phthalocyanine. Commonly used hole transporting polymers are polyvinylcarbazole, (phenylmethyl)-polysilane, and polyaniline. It is also possible to obtain hole transporting polymers by doping hole transporting molecules such as those mentioned above into polymers such as polystyrene and polycarbonate. In some cases, triarylamine polymers are used, especially triarylamine-fluorene copolymers. In some cases, the polymers and copolymers are crosslinkable. Examples of crosslinkable hole transport polymers can be found in, for example, published US patent application 2005-0184287 and published PCT application WO 2005/052027. In some embodiments, the hole transport layer is doped with a p-dopant, such as tetrafluorotetracyanoquinodimethane and perylene-3,4,9,10-tetracarboxylic-3,4,9,10-dianhydride.

In some embodiments, the electron transport layer 150 comprises the new deuterated compound of Formula I. Examples of other electron transport materials which can be used in layer 150 include metal chelated oxinoid compounds, such as tris(8-hydroxyquinolato)aluminum (Alq₃); bis(2-methyl-8-quinolinolato)(para-phenyl-phenolato)aluminum(III) (BAIQ); and azole compounds such as 2-(4-biphenylyl)-5-(4-t-butylphenyl)-1,3,4-oxadiazole (PBD) and 3-(4-biphenylyl)-4-phenyl-5-(4-t-butylphenyl)-1,2,4-triazole (TAZ), and 1,3,5-tri(phenyl-2-benzimidazole)benzene (TPBI); quinoxaline derivatives such as 2,3-bis(4-fluorophenyl)quinoxaline; phenanthroline derivatives such as 9,10-diphenylphenanthroline (DPA) and 2,9-dimethyl-4,7-diphenyl-1,10-phenanthroline (DDPA); and mixtures thereof. The electron-transport layer may also be doped with n-dopants, such as Cs or other alkali metals. Layer 150 can function both to facilitate electron transport, and also serve as a buffer layer or confinement layer to prevent quenching of the exciton at layer interfaces. Preferably, this layer promotes electron mobility and reduces exciton quenching.

The cathode 160, is an electrode that is particularly efficient for injecting electrons or negative charge carriers. The cathode can be any metal or nonmetal having a lower work function than the anode. Materials for the cathode can be selected from alkali metals of Group 1 (e.g., Li, Cs), the Group 2 (alkaline earth) metals, the Group 12 metals, including the rare earth elements and lanthanides, and the actinides. Materials such as aluminum, indium, calcium, barium, samarium and magnesium, as well as combinations, can be used. Li- or Cs-containing organometallic compounds, LiF, CsF, and Li₂O can also be deposited between the organic layer and the cathode layer to lower the operating voltage.

It is known to have other layers in organic electronic devices. For example, there can be a layer (not shown) between the anode 110 and buffer layer 120 to control the amount of positive charge injected and/or to provide band-gap matching of the layers, or to function as a protective layer. Layers that are known in the art can be used, such as copper phthalocyanine, silicon oxy-nitride, fluorocarbons, silanes, or an ultra-thin layer of a metal, such as Pt. Alternatively, some or all of anode layer 110, active layers 120, 130, 140, and 150, or cathode layer 160, can be surface-treated to increase charge carrier transport efficiency. The choice of materials for each of the component layers is preferably determined by balancing the positive and negative charges in the emitter layer to provide a device with high electroluminescence efficiency.

It is understood that each functional layer can be made up of more than one layer.

The device can be prepared by a variety of techniques, including sequential vapor deposition of the individual layers on a suitable substrate. Substrates such as glass, plastics, and metals can be used. Conventional vapor deposition techniques can be used, such as thermal evaporation, chemical vapor deposition, and the like. Alternatively, the organic layers can be applied from solutions or dispersions in suitable solvents, using conventional coating or printing techniques, including but not limited to spin-coating, dip-coating, roll-to-roll techniques, ink-jet printing, screen-printing, gravure printing and the like.

The present invention also relates to an electronic device comprising at least one active layer positioned between two electrical contact layers, wherein the at least one active layer of the device includes the anthracene compound of Formula I. Devices frequently have additional hole transport and electron transport layers.

To achieve a high efficiency LED, the HOMO (highest occupied molecular orbital) of the hole transport material desirably aligns with the work function of the anode, and the LUMO (lowest un-occupied molecular orbital) of the electron transport material desirably aligns with the work function of the cathode. Chemical compatibility and sublimation temperature of the materials are also important considerations in selecting the electron and hole transport materials.

It is understood that the efficiency of devices made with the anthracene compounds described herein, can be further improved by optimizing the other layers in the device. For example, more efficient cathodes such as Ca, Ba or LiF can be used. Shaped substrates and novel hole transport materials that result in a reduction in operating voltage or increase quantum efficiency are also applicable. Additional layers can also be added to tailor the energy levels of the various layers and facilitate electroluminescence.

The compounds of the invention often are fluorescent and photoluminescent and can be useful in applications other than OLEDs, such as oxygen sensitive indicators and as fluorescent indicators in bioassays.

### EXAMPLES

The following examples illustrate certain features and advantages of the present invention. They are intended to be illustrative of the invention, but not limiting. All percentages are by weight, unless otherwise indicated.

### Comparative Example A

This example illustrates the preparation of a non-deuterated compound, Comparative Compound A.

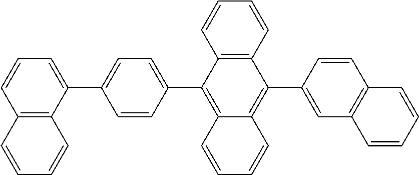

This compound can be prepared according to the following scheme:

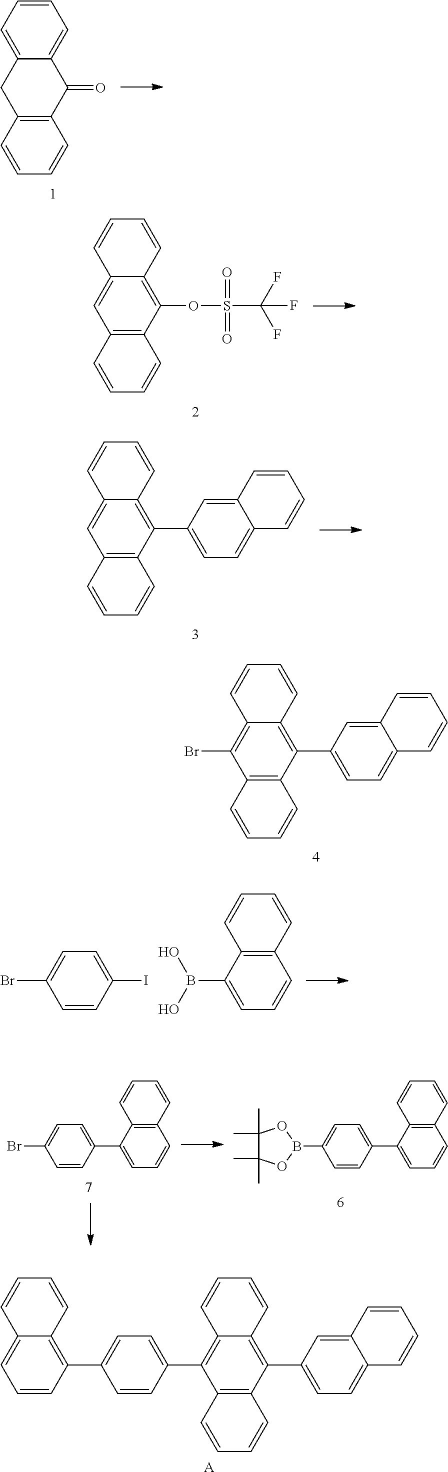

### Synthesis of Compound 2:

In a 3 L flask fitted with a mechanical stirrer, dropping funnel, thermometer and N₂ bubbler was added anthrone, 54 g (275.2 mmol) in 1.5 L dry methylene chloride. The flask was cooled in an ice bath and 1,8-diazabicyclo[5.4.0]undec-7-ene (“DBU”), 83.7 ml (559.7 mmol) was added by dropping funnel over 1.5 hr. The solution turned orange, became opaque, then turned deep red. To the still cooled solution was added triflic anhydride, 58 ml (345.0 mmol) via syringe over about 1.5 hr keeping the temperature of the solution below 5° C. The reaction was allowed to proceed for 3 hr at room temperature, after which 1 mL additional triflic anhydride was added and stirring at RT continued for 30 min. 500 mL water was added slowly and the layers separated. The aqueous layer was extracted with 3×200 mL dichloromethane (“DCM”) and the combined organics dried over magnesium sulfate, filtered and concentrated by rotary evaporation to give a red oil. Column chromatography on silica gel followed by crystallization from hexanes afforded 43.1 g (43%) of a tan powder

### Synthesis of Compound 3:

To a 200 mL Kjeldahl reaction flask equipped with a magnetic stirring bar in a nitrogen-filled glove box were added anthracen-9-yl trifluoromethanesulfonate (6.0 g, 18.40 mmol), Napthalen-2-yl-boronic acid (3.78 g 22.1 mmol), potassium phosphate tribasic (17.50 g, 82.0 mmol), palladium(II) acetate (0.41 g, 1.8 mmol), tricyclohexylphosphine (0.52 g, 1.8 mmol) and THF (100 mL). After removal from the dry box, the reaction mixture was purged with nitrogen and degassed water (50 mL) was added by syringe. A condenser was then added and the reaction was refluxed overnight. The reaction was monitored by TLC. Upon completion the reaction mixture was cooled to room temperature. The organic layer was separated and the aqueous layer was extracted with DCM. The organic fractions were combined, washed with brine and dried with magnesium sulfate. The solvent was removed under reduced pressure. The resulting solid was washed with acetone and hexane and filtered. Purification by column chromatography on silica gel afforded 4.03 g (72%) of product as pale yellow crystalline material.

### Synthesis of Compound 4:

9-(naphthalen-2-yl)anthracene, 11.17 g (36.7 mmol) was suspended in 100 mL DCM. N-bromosuccinimide 6.86 g (38.5 mmol) was added and the mixture stirred with illumination from a 100 W lamp. A yellow clear solution formed and then precipitation occurred. The reaction was monitored by TLC. After 1.5 h, the reaction mixture was partially concentrated to remove methylene chloride, and then crystallized from acetonitrile to afford 12.2 g of pale yellow crystals (87%).

### Synthesis of Compound 7:

To a 500 mL round bottom flask equipped with a stir bar in a nitrogen-filled glove box were added naphthalen-1-yl-1-boronic (14.2 g, 82.6 mmol), acid, 1-bromo-2-iodobenzene (25.8 g, 91.2 mmol), tetrakis(triphenylphospine) palladium(0) (1.2 g, 1.4 mmol), sodium carbonate (25.4 g, 240 mmol), and toluene (120 mL). After removal from the dry box, the reaction mixture was purged with nitrogen and degassed water (120 mL) was added by syringe. The reaction flask was then fitted with a condenser and the reaction was refluxed for 15 hours. The reaction was monitored by TLC. The reaction mixture was cooled to room temperature. The organic layer was separated and the aqueous layer was extracted with DCM. The organic fractions were combined and the solvent was removed under reduced pressure to give a yellow oil. Purification by column chromatography using silica gel afforded 13.6 g of a clear oil (58%).

### Synthesis of Compound 6:

To a 1-liter flask equipped with a magnetic stirring bar, a reflux condenser that was connected to a nitrogen line and an oil bath, were added 4-bromophenyl-1-naphthalene (28.4 g, 10.0 mmol), bis(pinacolate) diboron (40.8 g, 16.0 mmol), Pd(dppf)₂Cl₂ (1.64 g, 2.0 mmol), potassium acetate (19.7 g, 200 mmol), and DMSO (350 mL). The mixture was bubbled with nitrogen for 15 min and then Pd(dppf)₂Cl₂ (1.64 g, 0.002 mol) was added. During the process the mixture turned to a dark brown color gradually. The reaction was stirred at 120° C. (oil bath) under nitrogen for 18 h. After cooling the mixture was poured into ice water and extracted with chloroform (3×). The organic layer was washed with water (3×) and saturated brine (1×) and dried with MgSO₄. After filtration and removal of solvent, the residue was purified by chromatography on a silica gel column. The product containing fractions were combined and the solvent was removed by rotary evaporation. The resulting white solid was crystallized from hexane/chloroform and dried in a vacuum oven at 40° C. to give the product as white crystalline flakes (15.0 g in 45% yield). 1H and ¹³C-NMR spectra are in consistent with the expected structure.

### Synthesis of Comparative Compound A

To a 250 mL flask in glove box were added (2.00 g, 5.23 mmol), 4,4,5,5-tetramethyl-2-(4-(naphthalen-4-yl)phenyl)-1,3,2-dioxaborolane (1.90 g, 5.74 mmol), tris(dibenzylideneacetone) dipalladium(0) (0.24 g, 0.26 mmol), and toluene (50 mL). The reaction flask was removed from the dry box and fitted with a condenser and nitrogen inlet. Degassed aqueous sodium carbonate (2 M, 20 mL) was added via syringe. The reaction was stirred and heated to 90° C. overnight. The reaction was monitored by HPLC. After cooling to room temperature, the organic layer was separated. The aqueous layer was washed twice with DCM and the combined organic layers were concentrated by rotary evaporation to afford a grey powder. Purification by filtration over neutral alumina, hexanes precipitation, and column chromatography over silica gel afforded 2.28 g of a white powder (86%).

The product was further purified as described in published U.S. patent application 2008-0138655, to achieve an HPLC purity of at least 99.9% and an impurity absorbance no greater than 0.01.

Alternatively, Compound A can be synthesized from commercial starting materials according to the process scheme illustrated below:

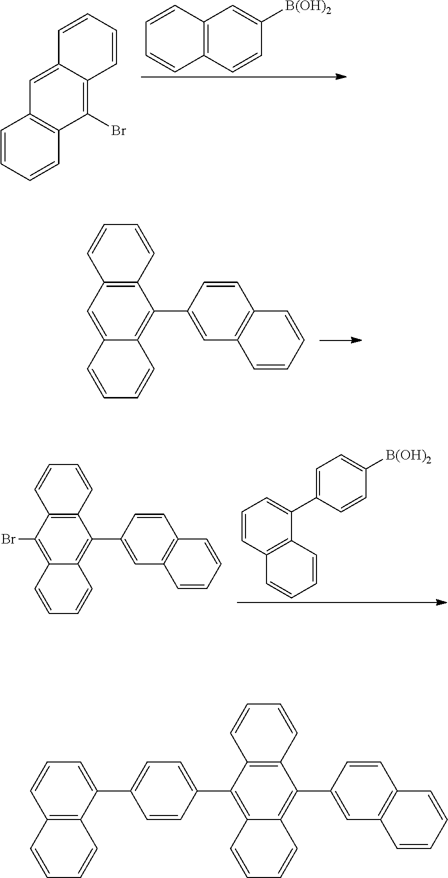

### Example 1

This example illustrates the preparation of a compound having Formula I, Compound H1.

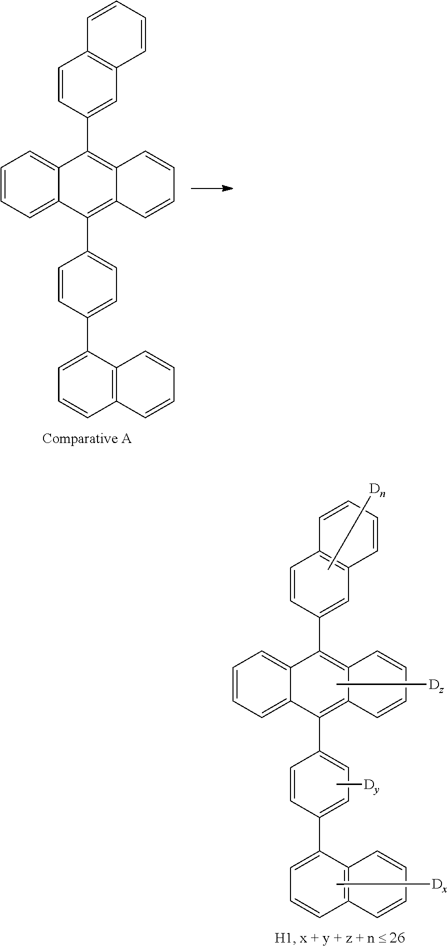

Under an atmosphere of nitrogen, AlCl₃ (0.48 g, 3.6 mmol) was added to a perdeuterobenzene or benzene-D6 (C₆D₆) (100 mL) solution of comparative compound A from Comparative Example A (5 g, 9.87 mmol). The resulting mixture was stirred at room temperature for six hours after which D₂O (50 mL) was added. The layers were separated followed by washing the water layer with CH₂Cl₂ (2×30 mL). The combined organic layers were dried over magnesium sulfate and the volatiles were removed by rotary evaporation. The crude product was purified via column chromatography. The deuterated product H1 (x+y+n+m=21-23) was obtained (4.5 g) as a white powder.

The product was further purified as described in published U.S. patent application 2008-0138655, to achieve an HPLC purity of at least 99.9% and an impurity absorbance no greater than 0.01. The material was determined to have the same level of purity as comparative compound A, from above.

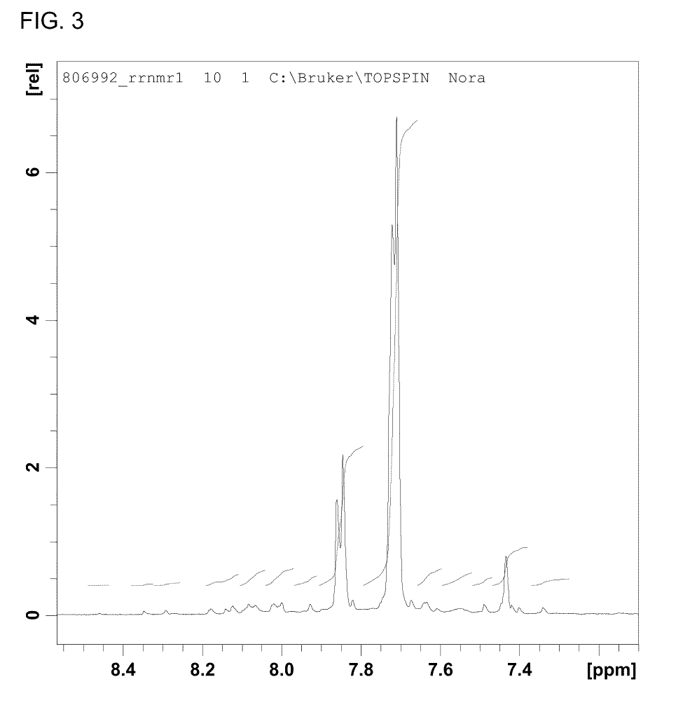

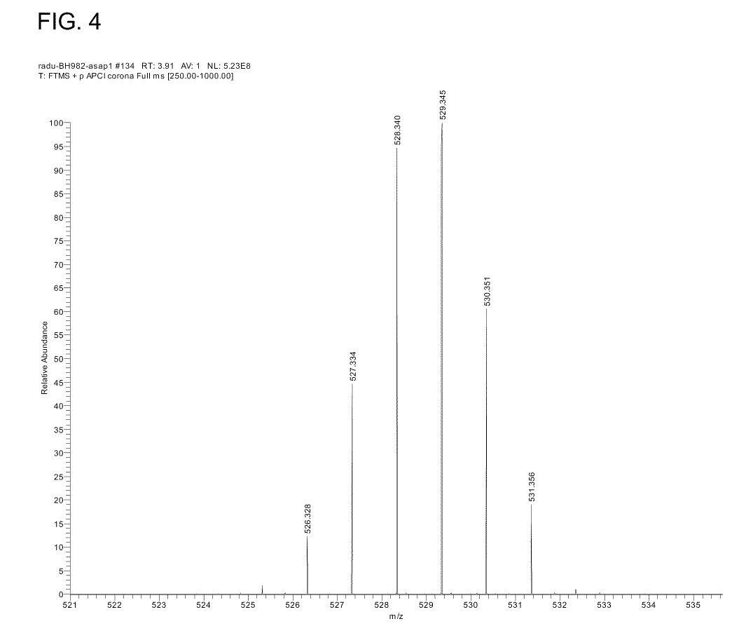

The ¹H NMR (CD₂Cl₂) and ASAP-MS are shown in FIGS. 3 and 4, respectively. The compound had the structure given below:

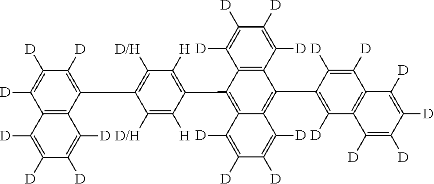

where “D/H” indicates approximately equal probability of H or D at this atomic position. The structure was confirmed by ¹H NMR, ¹³C NMR, ²D NMR and ¹H—¹³C HSQC (Heteronuclear Single Quantum Coherence).

### Examples 2 and 3 and Comparative Examples B and C

These examples demonstrate the fabrication and performance of a device with a blue emitter. The following materials were used:

### Emitter E1

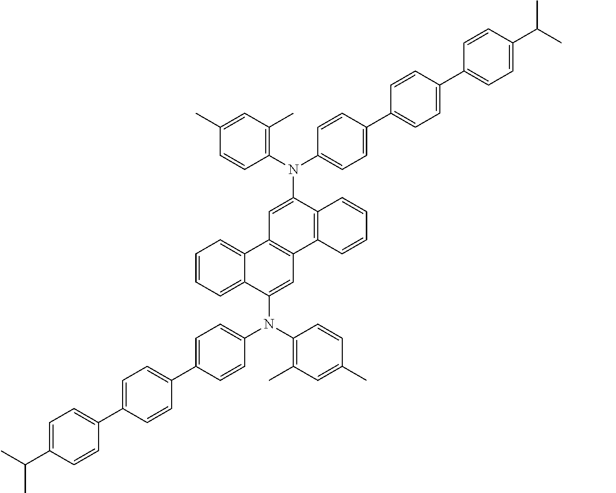

### Emitter E2:

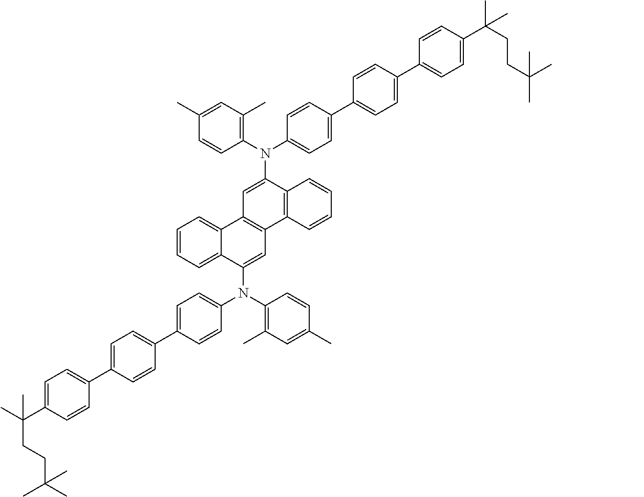

The device had the following structure on a glass substrate:

anode=Indium Tin Oxide (ITO): 50 nm

buffer layer=Buffer 1 (50 nm), which is an aqueous dispersion of an electrically conductive polymer and a polymeric fluorinated sulfonic acid. Such materials have been described in, for example, published U.S. patent applications US 2004/0102577, US 2004/0127637, and US 2005/0205860.

hole transport layer=polymer P1, which is a non-crosslinked arylamine polymer (20 nm)

photoactive layer=13:1 host:dopant (40 nm), as shown in Table 1

electron transport layer=a metal quinolate derivative (10 nm)

cathode=CsF/Al (1.0/100 nm)

TABLE 1
Device Photoactive Layers

| Example | Host | Dopant |
|---|---|---|
| Comparative B-1 | Comp. Compound A | E1 |
| Comparative B-2 | Comp. Compound A | E1 |
| Comparative B-3 | Comp. Compound A | E1 |
| Comparative B-4 | Comp. Compound A | E1 |
| Ex. 2-1 | H1 | E1 |
| Ex. 2-2 | H1 | E1 |
| Ex. 2-3 | H1 | E1 |
| Ex. 2-4 | H1 | E1 |
| Comparative C-1 | Comp. Compound A | E2 |
| Comparative C-2 | Comp. Compound A | E2 |
| Ex. 3-1 | H1 | E2 |
| Ex. 3-2 | H1 | E2 |

OLED devices were fabricated by a combination of solution processing and thermal evaporation techniques. Patterned indium tin oxide (ITO) coated glass substrates from Thin Film Devices, Inc were used. These ITO substrates are based on Corning 1737 glass coated with ITO having a sheet resistance of 30 ohms/square and 80% light transmission. The patterned ITO substrates were cleaned ultrasonically in aqueous detergent solution and rinsed with distilled water. The patterned ITO was subsequently cleaned ultrasonically in acetone, rinsed with isopropanol, and dried in a stream of nitrogen.

Immediately before device fabrication the cleaned, patterned ITO substrates were treated with UV ozone for 10 minutes. Immediately after cooling, an aqueous dispersion of Buffer 1 was spin-coated over the ITO surface and heated to remove solvent. After cooling, the substrates were then spin-coated with a solution of a hole transport material, and then heated to remove solvent. After cooling the substrates were spin-coated with the emissive layer solution, and heated to remove solvent. The substrates were masked and placed in a vacuum chamber. The electron transport layer was deposited by thermal evaporation, followed by a layer of CsF. Masks were then changed in vacuo and a layer of Al was deposited by thermal evaporation. The chamber was vented, and the devices were encapsulated using a glass lid, dessicant, and UV curable epoxy.

The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current needed to run the device. The unit is a cd/A. The power efficiency is the current efficiency multiplied by pi, divided by the operating voltage. The unit is Im/W. The device data is given in Table 2.

TABLE 2
Device Performance

| | | | | | | Lifetest | | | |
|---|---|---|---|---|---|---|---|---|---|
| | | | | | | current | Lifetest | Raw | Projected |
| | CIE | Voltage | C.E. | E.Q.E. | P.E. | density | Luminance | T50 | Lifetime T50 |
| Ex. | (x, y) | (V) | (cd/A) | (%) | (lm/W) | (mA/cm2) | (nits) | (h) | @1000 nits |
| Comp. | 0.135, | 4.4 | 6.1 | 6.0 | 4.3 | 129 | 6837 | 410 | 10766 |
| B-1 | 0.124 | | | | | | | | |
| Comp. | 0.135, | 4.3 | 6.4 | 6.1 | 4.7 | 125 | 7002 | 410 | 11211 |
| B-2 | 0.127 | | | | | | | | |
| Comp. | 0.136, | 4.4 | 5.7 | 5.6 | 4.1 | 127 | 5906 | 430 | 8804 |
| B-3 | 0.123 | | | | | | | | |
| Comp. | 0.134, | 4.3 | 6.4 | 6.1 | 4.7 | 123 | 7021 | 430 | 11813 |
| B-4 | 0.129 | | | | | | | | |
| Ex. | 0.136, | 4.4 | 5.7 | 5.7 | 4.1 | 136 | 7040 | 870 | 24010 |
| 2-1 | 0.119 | | | | | | | | |
| Ex. | 0.137, | 4.2 | 5.4 | 5.5 | 4.1 | 126 | 6244 | 830 | 18679 |
| 2-2 | 0.117 | | | | | | | | |
| Ex. | 0.136, | 4.2 | 5.7 | 5.7 | 4.2 | 129 | 6467 | 830 | 19828 |
| 2-3 | 0.118 | | | | | | | | |
| Ex. | 0.136, | 4.3 | 5.5 | 5.6 | 4.1 | 121 | 6148 | 870 | 19071 |
| 2-4 | 0.118 | | | | | | | | |
| Comp. | 0.135, | 4.3 | 6.2 | 6.0 | 4.6 | 141 | 8018 | 490 | 16871 |
| C-1 | 0.128 | | | | | | | | |
| Comp. | 0.135, | 4.3 | 6.3 | 6.0 | 4.6 | 125 | 7136 | 510 | 14403 |
| C-2 | 0.128 | | | | | | | | |
| Ex. | 0.135, | 4.3 | 6.0 | 5.9 | 4.4 | 121 | 6741 | 950 | 24356 |
| 3-1 | 0.122 | | | | | | | | |
| Ex. | 0.135, | 4.2 | 6 | 5.8 | 4.4 | 126 | 6974 | 930 | 25257 |
| 3-2 | 0.124 | | | | | | | | |

* All data @ 1000 nits, CE = current efficiency; CIEx and CIEy are the x and y color coordinates according to the C.I.E. chromaticity scale (Commission Internationale de L'Eclairage, 1931). RawT50 is the time in hours for a device to reach one-half the initial luminance at the lifetest luminance given. Projected T50 is the projected lifetime at 1000 nits using an accelerator factor of 1.7.

It can be seen that with the deuterated host of the invention, the lifetime of devices is greatly increased. When emitter E1 was used, the comparative devices with a non-deuterated host (Comparative examples B-1 through B-4) had an average raw T50 of 420 hours. With the deuterated analog host H1 (Examples 2-1 through 2-4), the devices has an average raw T50 of 850 hours. When emitter E2 was used, the comparative devices (C-1 and C-2) had an average raw T50 of 500 hours. With the deuterated analog host H1 (3-1 and 3-2), the average raw T50 was 940 hours.

### Example 4

This example illustrates the preparation of some deuterated intermediate compounds that can be used to synthesize compounds having Formula I with controlled levels of deuteration.

### Intermediate A:

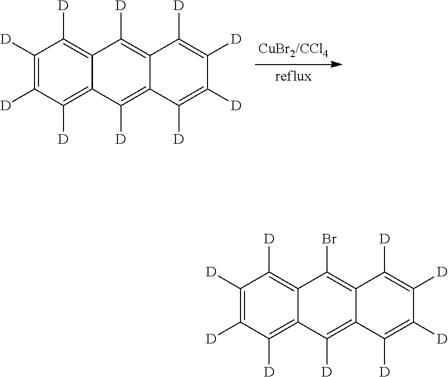

To a solution of anthracene-d10 (18.8 g, 0.10 mole) in 0014 (500 mL) was added anhydrous cupric bromide (45 g, 0.202 mole) in one portion. The reaction mixture was stirred and heated under reflux for 12 hours. The brown cupric chloride is gradually converted to white cuprous bromide, and hydrogen bromide is gradually evolved (connected to base bath absorber). At the end of the reaction the cuprous bromide was removed by filtration, and the carbon tetrachloride solution was passed through a 35-mm. Chromatographic column filled with 200 g. of alumina. The column is eluted with 200 ml of CH₂Cl₂. The combined eluates are evaporated to dryness to give 24 g. (87%) of 9-bromoanthracene-d9 as a lemon-yellow solid. It contains impurity of the starting material (˜2%) and the dibromo-byproduct (˜2%). This material was used directly in further coupling reactions without purification. The intermediate can be further purified to by recrystallization using hexane or cyclohexane to give the pure compound.

### Intermediate B:

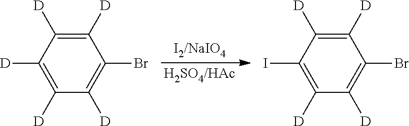

To d5-bromobenzene (MW 162, 100 g, 0.617 mol), was added a mixture solvents of 93 mL of 50% H2SO4, and 494 mL of HOAc at rt. Then a pulverized I2 (MW 254, 61.7 g, 0.243 mol) was added followed by pulverized NaIO4 (MW 214, 26.4 g, 0.123 mol). The mixture was vigorously stirred and heated to 90° C. for 4 h. The dark purple color solution changed to a pale-orange-colored mixture containing a very fine white precipitate. The mixture was allowed to cool to rt overnight. During this time, the product precipitated as microcrystalline plates. The mixture was filtered and was washed twice 10% sodium thiosulfate Na2S2O3 (50 mL) and then with water. It was dissolved in CH2Cl2 and run flash column. The light yellow, crystalline material was obtained 124 g (70%). The filtrate was extracted with CH₂Cl₂ (50 mL×3) and combined the CH2Cl2 washed twice 10% sodium thiosulfate Na2S2O3 (50 mL) and then with water. After dried and evaporated the solvent and run flash column to give another 32 g of pure product (17.5%). Total is 156 g (yield 88%).

### Intermediate C:

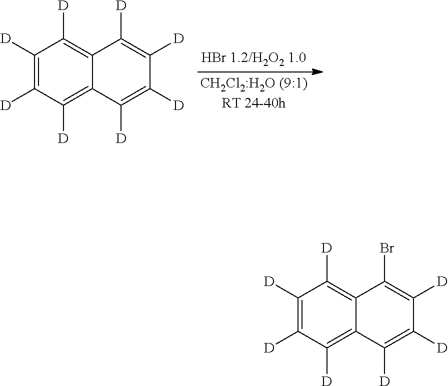

To a stirred solution of naphalene-d8 (MW 136, 68 g, 0.5 mole) in CH₂Cl2 (800 mL): H20 (80 mL) and hydrobromic acid (MW: 81, d=1.49, 100 g; 67.5 mL of a 49% aq. solution; 0.6 mol) was slowly added hydrogen peroxide (FW: 34, d=1.1 g/mL, 56 g; 51.5 mL of a 30% aq. solution; 0.5 mol) over a period of 30 min at 10-15° C. The reaction was left at room temperature for 40 h whilst monitoring its progress by TLC. After the completion of bromination, the solvent was removed under reduced pressure and the crude product was washed twice 10% sodium thiosulfate Na2S2O3 (50 mL) and then with water. The pure product was isolated by flash column chromatography on silica gel (100-200 mesh) using hexane (100%) followed by distillation to give pure 1-bromo-naphthene-d7 as a clear liquid 85 g, the yield is around 80%.

### Intermediate D:

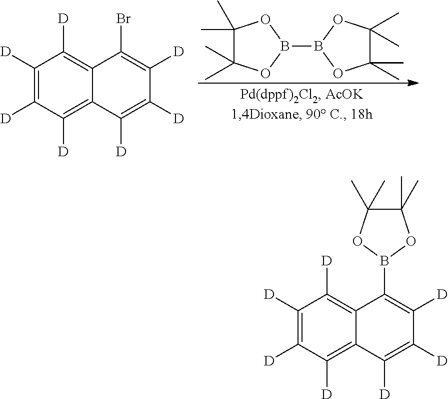

The mixture of 1-bromonaphthalene-d7 (21.4 g, 0.10 mol), bis(pinacolato)diboron (38 g, 0.15 mol), potassium acetate (19.6 g, 0.20 mol) in 300 ml of dry 1,4-dioxane was bubbled with nitrogen for 15 min. Then Pd(dppf)₂Cl₂-CH₂Cl₂(1.63 g, 0.002 mol) was added. The mixture was heated at 100° C. (oil bath) for 18 h. After cooling down the mixture was filtered through CELIT and then concentrated to 50 mL, then added water and extracted with ether for three times (100 mL×3). The organic layer was washed with water (3×) and brine (1×), dried over MgSO₄, filtered and concentrated. The residue was submitted to a silica gel column (eluent: hexane) to give a white liquid which has by products of naphalene, and diboronic ester. Thus further purification was conducted by distilliation to give a viscous clear liquid. Yield 21 g, 82%.

### Intermediate E:

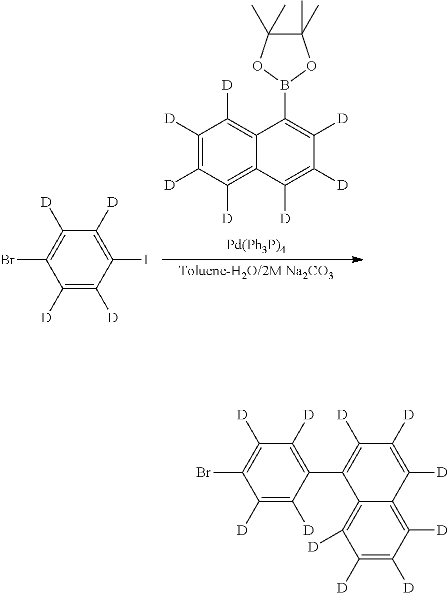

To a mixture of 1-bromo-4-iodo-benzene-D4 (10.95 g, 0.0382 mole) and 1-naphaleneboronic ester-D7 (10.0 g, 0.0383 mole) in Toluene (300 mL) was added Na₂CO₃ (12.6 g, 0.12 mole) and H2O (50 mL), aliquant (3 g). The mixture was bubbled with nitrogen for 15 min. Then Pd(PPh₃)4 (0.90 g, 2%) was added. The mixture was refluxed for 12 h under a nitrogen atmosphere. After cooling down the reaction mixture was separated, the organic layer was washed with water and separated, dried and concentrated. Silica was added and concentrated. After evaporation the residue solvent, it was subject to run flash column using hexane as eluent to give crude product. Further purification was conducted by distillation (collect 135-140° C./100 mtorr) to give clear viscous liquid (8.76 g, yield 78%).

### Intermediate F:

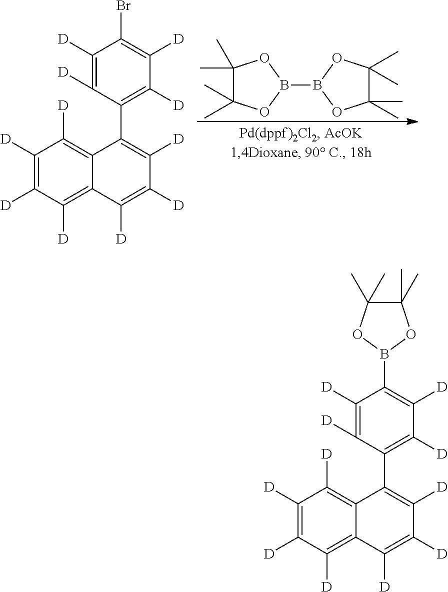

The mixture of 1-bromo-phenyl-4-naphthalene-d11 (22 g, 0.075 mole), bis(pinacolato)diboron (23 g, 0.090 mol), potassium acetate 22 g, 0.224 mol) in 200 ml of dry 1,4-dioxane was bubbled with nitrogen for 15 min. Then Pd(dppf)₂Cl₂.CH₂Cl₂(1.20 g, 0.00147 mol) was added. The mixture was heated at 100° C. (oil bath) for 18 h. After cooling down the mixture was filtered through CELIT and then concentrated to 50 mL, then added water and extracted with ether for three times (100 mL×3). The organic layer was washed with water (3×) and brine (1×), dried over MgSO₄, filtered and concentrated. The residue was submitted to a silica gel column (eluent: hexane) to give a white liquid which has by products of naphalene, and diboronic ester. Thus further purification was conducted by run silica gel column again using hexane as eluent. After evaporate the solvent and concentrated to around 80 mL hexane and white crystal product was formed, it was filtrate to give 20.1 g of product, yield 81%.

### Intermediate G:

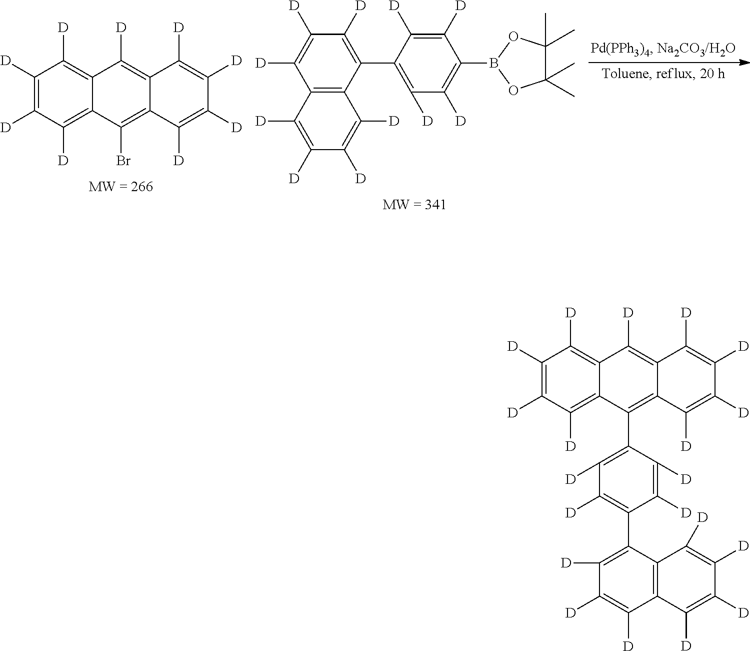

To the intermediate A (18.2 g) and intermediate F boronic ester (25.5 g) in Toluene (500 mL) was added Na₂CO₃ (31.8 g) and H2O (120 mL), aliquant (5 g). The mixture was bubbled with nitrogen for 15 min. Then Pd(PPh3)₄ (1.5 g, 1.3%) was added. The mixture was refluxed for 12 h under a nitrogen atmosphere. After cooling down the reaction mixture was separated, the organic layer was washed with water and separated, dried and concentrated to ˜50 mL and poured into MeOH. The solid was filtered to give a yellow crude product (˜28.0 g). The crude product was washed with water, HCl (10%), water and methanol. It was redissolved in CHCl₃, dried over MgSO4, filtered. The filtrate was added silica gel, concentrated and dried, purified on silica gel (0.5 Kg) using hexane only as eluent (total of 50 L hexane passed—recycled using only 5 L of hexane) to give the white product.

### Intermediate H:

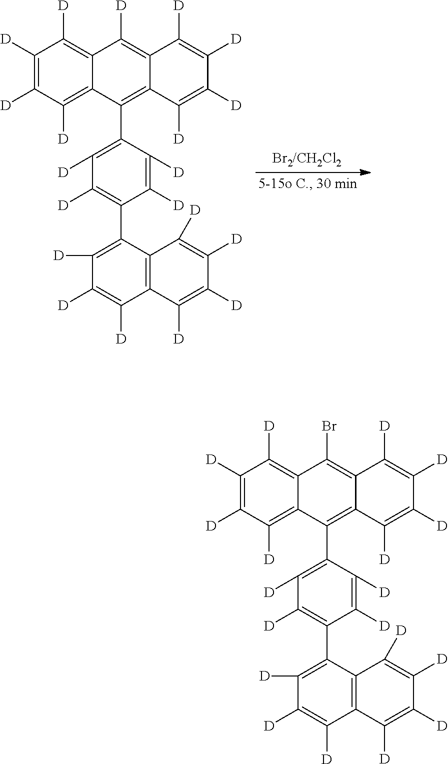

Into a ice-bath cooled solution of 9-(4-naphthalen-1-yl)phenylanthracene-D20 Intermediate G (MW 400.6, 20.3 g, 0.05 mole) in CH2Cl2 (450 mL) was added slowly (20 min) of bromine (MW 160, 8.0 g, 0.05 mole) dissolved in CH2Cl2 (150 mL). The reaction immediately occurred and the color changed to light yellow. Add a solution of Na2S2O3 (2M 100 mL) and stirred for 15 min. Then separated the water layer and the organic phase was washed by Na2CO3 (10%, 50 mL), followed by three times of water. Separated and then dried by MgSO4 and after evaporated the solvent till 100 mL left. Powered into methanol (200 mL) and filtered to give 23.3 g of pure compound (MW 478.5, yield. 97.5%) HPLC shows 100% purity.

### Intermediate I:

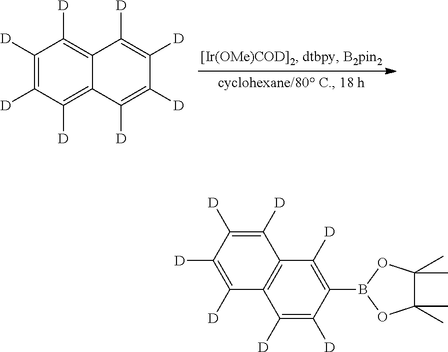

The mixture of naphthalene-D8 (13.6 g, 0.10 mole), bis(pinacolato)diboron (27.93 g, 0.11 mole), di-mu-methoxobis(1,5-cyclooctadiene)diiradium (I) [Ir(OMe)COD]₂ (1.35 g, 2 mmole, 2%) and 4,4′-di-tert-butyl-2,2′-bipyridine (1.1 g, 4 mmole) was added to cyclohexane (200 mL). The mixture was degassed with N2 for 15 min, then heated at 85° C. (oil bath) overnight (dark brown solution). The mixture was passed through a pad of silica gel. The fractions were collected and concentrated until dry. Hexane was added. The filtrate was concentrated (liquid) and passed through a silica gel column, rinsing with hexane to give clear liquid, it was not pure and was purified again by silica gel column, rinsing with hexane followed by distillation at 135° C./100 mmtorr to give pure white viscous liquid and it solidified to give a white powder (18.5 g. Yield 70%).

### Intermediate J:

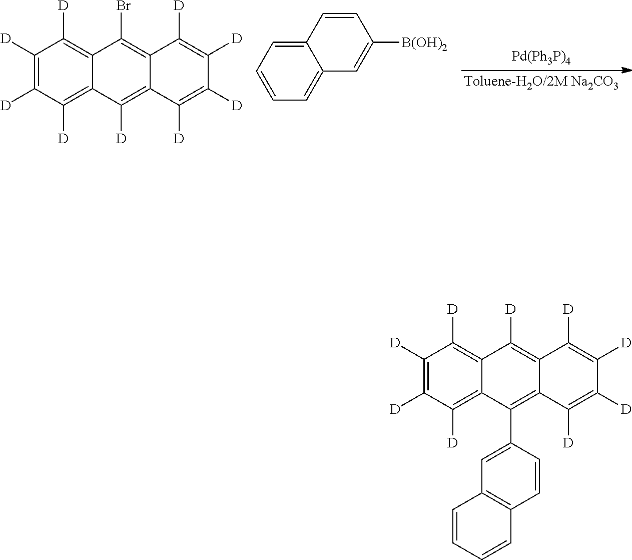

Into a RBF (100 mL) was added 9-bromoanthracene-d9 (MW 266, 2.66 g, 0.01 mole), naphthelen-2-boronic acid(MW 172, 1.72 g, 0.01 mol), followed by the addition of toluene (30 mL), The mixture was purged with N₂ for 10 min. Then Na₂CO₃ (2M, 10 mL (2.12 g) 0.02 mole) dissolved in the water (10 mL) was added. The mixture was continued to purge with N₂ for 10 min. A catalyst amount of Pd(PPh₃)₄ (0.25 g, 2.5%, 0.025 mmol) was added. The mixture was refluxed overnight. Separated the organic layer then poured into metahol, washed with water, HCl (10%), water and methanol. It gives 2.6 g pure white product. (Yield: 83%).

### Intermediate K:

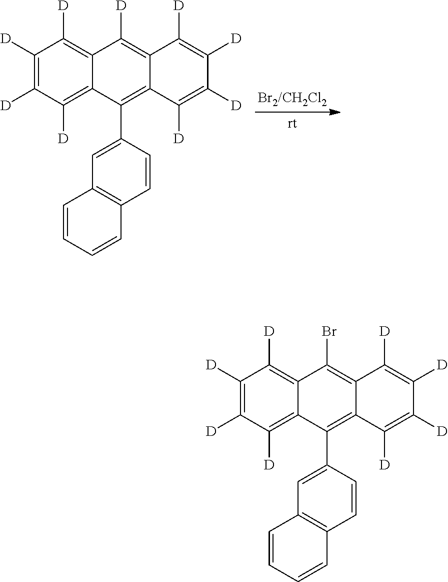

A solution of (2.6 g 0.0083 mole)9-2′-naphthyl-anthacene-d9 intermediate J in CH2Cl2 (50 mL) was added dropwise a solution of bromine (1.33 g, 0.0083 mole) in CH2Cl2 (5 mL) and stirred for 30 min. Add a solution of Na2S2O3 (2M 10 mL) and stirred for 15 min. Then separated the water layer and the organic phase was washed by Na₂CO₃ (10%, 10 mL), followed by three times of water. Separated and then dried by MgSO4 and after evaporated the solvent till 20 mL left. Powered into methanol (100 mL) and filtered give pure compound (3.1 g, yield 96%).

### Intermediate L:

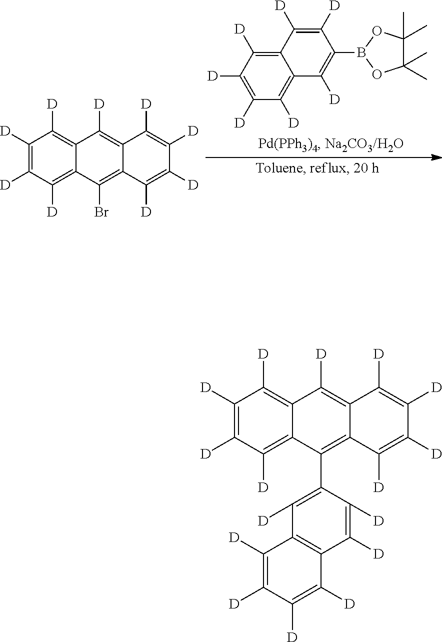

To a mixture of 9-bromoanthracene-D9 intermediate K (2.66 g, 0.01 mole) and 4,4,5,5-tetramethyl-2-(naphthalene-2-yl-D7)-1,3,2-dioxaborolane (2.7 g, 0.011 mole) in Toluene (˜60 mL) was added Na₂CO₃ (4.0 g, 0.04 mole) and H2O (20 mL). The mixture was bubbled with nitrogen for 15 min. Then Pd(PPh₃)₄ (0.20 g, 2.0%) was added. The mixture was refluxed for 18 h under a nitrogen atmosphere (yellow solids). After cooling down the reaction mixture, it was poured into MeOH (200 mL). The solid was filtered to give a yellow crude product. The crude product was washed with water, and methanol. It was redissolved in CHCl₃, dried over MgSO4, filtered. The filtrate was added silica gel, concentrated and dried, purified on silica gel using hexane as eluent to give the pure product (3.0 g, yield 94%).

### Intermediate M:

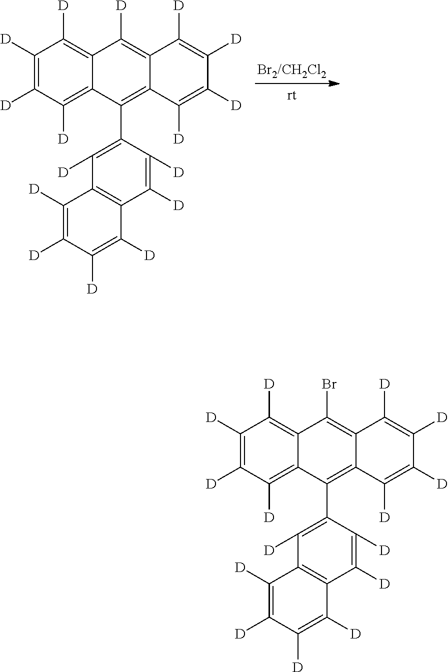

A solution of 9-2′-naphthyl-anthacene-d9 intermediate L (2.8 g 0.00875 mole) in CH₂Cl₂ (50 mL) was added dropwise a solution of bromine (1.4 g, 0.00875 mole) in CH₂Cl₂ (5 mL) and stirred for 30 min. Then a solution of Na₂S₂O₃ (2M 10 mL) was added and the mixture was stirred for 15 min. Then separated the water layer and the organic phase was washed by Na₂CO₃ (10%, 10 mL), followed by three times of water. Separated and then dried by MgSO₄ and after evaporated the solvent till 20 mL left. Powered into methanol (100 mL) and filtered give pure compound (3.3 g, yield 95%).

### Example 5

This example illustrates the synthesis of Compound H8 from Intermediate H and Intermediate I.

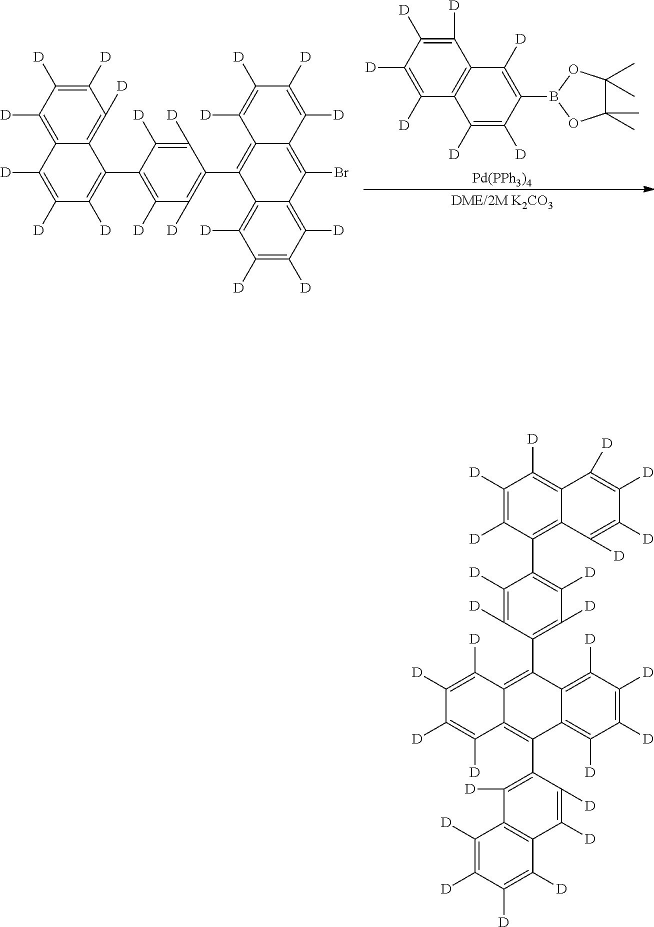

To a mixture of 9bromo-10-(4-naphthalen-1-yl)phenylanthracene-D19 intermediate H (14.84 g, 0.031 mole) and 2-naphthalen boronic ester intermediate I (10.0 g, 0.038 mole) in DME (350 mL) was added K₂CO₃ (12.8 g, 0.093 mole) and H2O (40 mL). The mixture was bubbled with nitrogen for 15 min. Then Pd(PPh₃)₄ (0.45 g, 1.3%) was added. The mixture was refluxed for 12 h under a nitrogen atmosphere. After cooling down the reaction mixture was concentrated to ˜150 mL and poured into MeOH. The solid was filtered to give a light yellow crude product. The crude product was washed with water, and methanol. It was redissolved in CHCl₃, dried over MgSO₄, filtered. The filtrate was added silica gel, concentrated and dried, purified on silica gel (0.5 Kg) using hexane:chloroform (3:1) as eluent to give the white product. (15 g, yield 91%)

### Example 6

This example illustrates the synthesis of Compound H13 from Intermediate K.

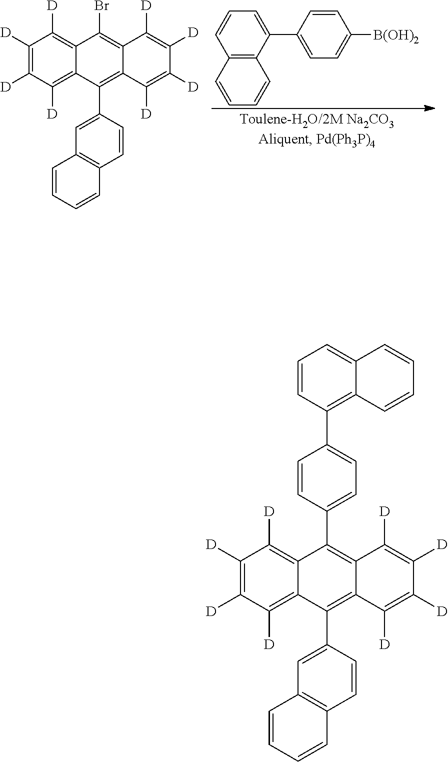

Into a RBF (100 mL) was added 9-bromo-10-(naphthalene-2-yl)anthracene intermediate K (1.96 g, 0.05 mol), 4-(naphthalene-1-yl)phenylboronic acid (1.49 g, 0.06 mol), followed by the addition of toluene (30 mL). The mixture was purged with N2 for 10 min. Then Na₂CO₃ (1.90 g, 0.018 mole) dissolved in the water (8 mL) was added, followed by Aliquent (1 mL). The mixture was continued to purge with N2 for 10 min. A catalyst amount of Pd(PPh₃)₄ (116 mg) was added. The mixture was refluxed overnight. After split of aqueous phase, organic layer was poured into methanol (100 mL) to collect the white solid. It was filtrated and further purification was conducted by running the silica gel column using chloroform:hexane (1:3) to give pure white compound (2.30 g, yield 90%).

### Example 7

This example illustrates the synthesis of Compound H9 from Intermediate I and Intermediate F.

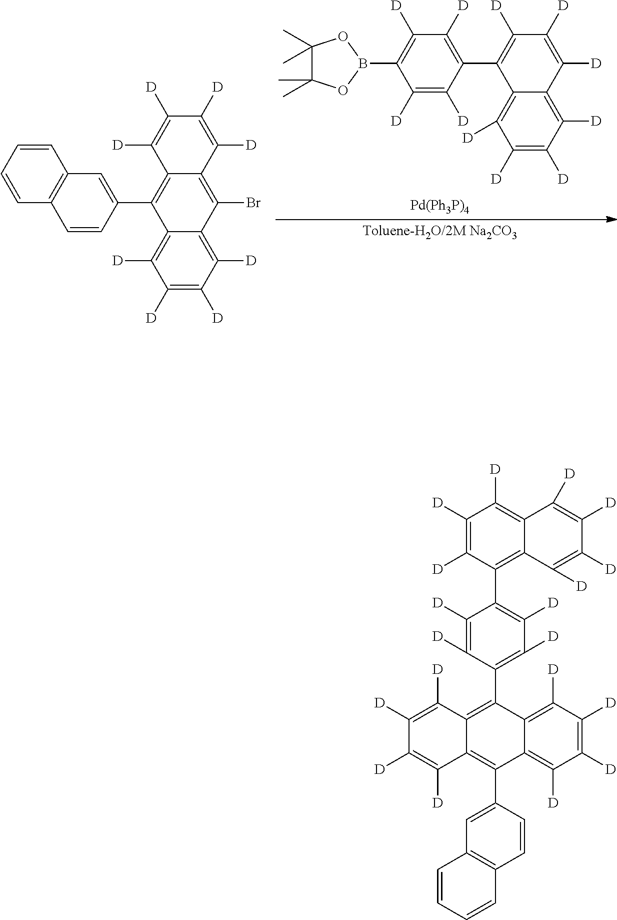

Into a RBF (100 mL) was added 9-bromo-10-(naphthalene-2-yl)anthracene-D8 intermediate K (0.70 g, 0.0018 mol), 4-(naphthalene-1-yl)phenylboronic acid-D11 intermediate F (0.7 g, 0.002 mol), followed by the addition of toluene (10 mL). The mixture was purged with N₂ for 10 min. Then Na₂CO₃ (0.64 g, 0.006 mole) dissolved in the water (3 mL) was added, followed by Aliquent 0.1 mL). The mixture was continued to purge with N₂ for 10 min. A catalyst amount of Pd(PPh₃)₄ (0.10 g) was added. The mixture was refluxed overnight. After split of aqueous phase, organic layer was poured into methanol (100 mL) to collect the white solid. It was filtrated and further purification was conducted by running the silica gel column using chloroform:hexane (1:3) to give pure white compound (0.90 g, yield 95%).

Compounds H10, H11 and H12 were prepared in an analogous manner.

### Examples 8-10 and Comparative Examples D and E

These examples demonstrate the fabrication and performance of a device with a blue emitter. The following materials were used:

### Emitter E3:

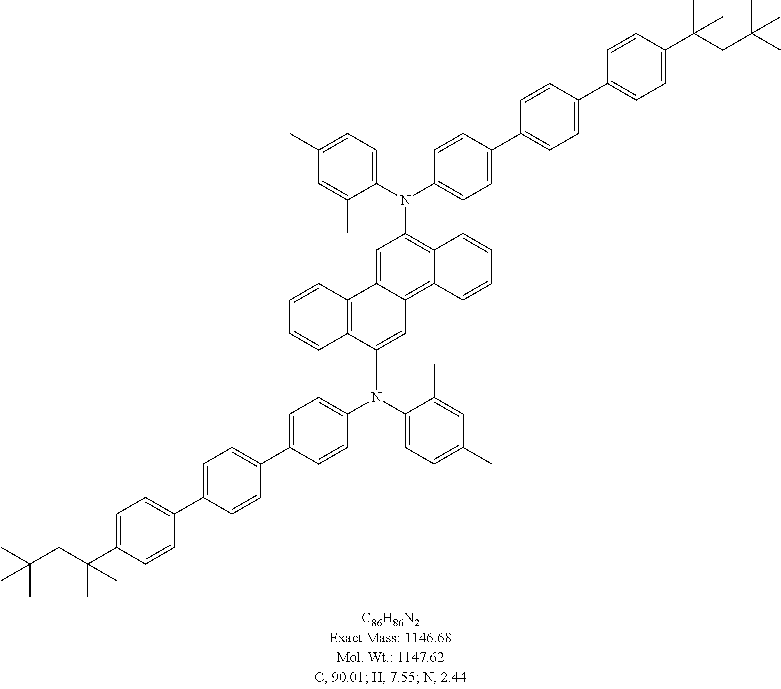

The device had the following structure on a glass substrate:

anode=ITO (50 nm)

buffer layer=Buffer 1 (50 nm).

hole transport layer=polymer P1 (20 nm)

photoactive layer=13:1 host:dopant (40 nm), as shown in Table 3

electron transport layer=a metal quinolate derivative (10 nm)

cathode=CsF/Al (1.0/100 nm)

TABLE 3
Device Photoactive Layers

| Example | Host | Dopant |
|---|---|---|
| Comparative D-1 | Comp. Compound A | E3 |
| Comparative D-2 | Comp. Compound A | E3 |
| Ex. 8-1 | H11 | E3 |
| Ex. 8-2 | H11 | E3 |
| Ex. 9-1 | H8 | E3 |
| Ex. 9-2 | H8 | E3 |
| Comparative E-1 | Comp. Compound A | E3 |
| Comparative E-2 | Comp. Compound A | E3 |
| Ex. 10-1 | H10 | E3 |
| Ex. 10-2 | H10 | E3 |

OLED devices were fabricated by a combination of solution processing and thermal evaporation techniques. Patterned indium tin oxide (ITO) coated glass substrates from Thin Film Devices, Inc were used. These ITO substrates are based on Corning 1737 glass coated with ITO having a sheet resistance of 30 ohms/square and 80% light transmission. The patterned ITO substrates were cleaned ultrasonically in aqueous detergent solution and rinsed with distilled water. The patterned ITO was subsequently cleaned ultrasonically in acetone, rinsed with isopropanol, and dried in a stream of nitrogen.

Immediately before device fabrication the cleaned, patterned ITO substrates were treated with UV ozone for 10 minutes. Immediately after cooling, an aqueous dispersion of Buffer 1 was spin-coated over the ITO surface and heated to remove solvent. After cooling, the substrates were then spin-coated with a solution of a hole transport material, and then heated to remove solvent. After cooling the substrates were spin-coated with the emissive layer solution, and heated to remove solvent. The substrates were masked and placed in a vacuum chamber. The electron transport layer was deposited by thermal evaporation, followed by a layer of CsF. Masks were then changed in vacuo and a layer of Al was deposited by thermal evaporation. The chamber was vented, and the devices were encapsulated using a glass lid, dessicant, and UV curable epoxy.

The OLED samples were characterized by measuring their (1) current-voltage (I-V) curves, (2) electroluminescence radiance versus voltage, and (3) electroluminescence spectra versus voltage. All three measurements were performed at the same time and controlled by a computer. The current efficiency of the device at a certain voltage is determined by dividing the electroluminescence radiance of the LED by the current needed to run the device. The unit is a cd/A. The power efficiency is the current efficiency multiplied by pi, divided by the operating voltage. The unit is Im/W. The device data is given in Table 4.

TABLE 4
Device Performance

| | | | | | | Lifetest | | | |
|---|---|---|---|---|---|---|---|---|---|
| | | | | | | current | Lifetest | Raw | Projected |
| | CIE | Voltage | C.E. | E.Q.E. | P.E. | density | Luminance | T50 | Lifetime T50 |
| Ex. | (x, y) | (V) | (cd/A) | (%) | (lm/W) | (mA/cm2) | (nits) | (h) | @1000 nits |
| Comp. | 0.136, | 4.4 | 5.7 | 5.5 | 4.1 | 127 | 6686 | 455 | 11503 |
| D-1 | 0.126 | | | | | | | | |
| Comp. | 0.136, | 4.4 | 5.8 | 5.7 | 4.2 | 129 | 6572 | 450 | 11048 |
| D-2 | 0.123 | | | | | | | | |
| Ex. | 0.136, | 4.4 | 5.8 | 5.8 | 4.2 | 128 | 6669 | 555 | 13970 |
| 8-1 | 0.121 | | | | | | | | |
| Ex. | 0.136, | 4.3 | 5.9 | 5.7 | 4.2 | 130 | 6599 | 590 | 14587 |
| 8-2 | 0.124 | | | | | | | | |
| Ex. | 0.136, | 4.3 | 5.8 | 5.8 | 4.2 | 127 | 6475 | 940 | 22503 |
| 9-1 | 0.121 | | | | | | | | |
| Ex. | 0.136, | 4.4 | 5.8 | 5.8 | 4.2 | 124 | 6254 | 955 | 21551 |
| 9-2 | 0.121 | | | | | | | | |
| Comp. | 0.136, | 4.4 | 6.1 | 6.0 | 4.3 | 124 | 6670 | 400 | 10071 |
| E-1 | 0.123 | | | | | | | | |
| Comp. | 0.135, | 4.4 | 5.8 | 5.6 | 4.1 | 131 | 7006 | 388 | 10620 |
| E-2 | 0.124 | | | | | | | | |
| Ex. | 0.136, | 4.4 | 6.0 | 5.9 | 4.2 | 123 | 6403 | 622 | 14610 |
| 10-1 | 0.121 | | | | | | | | |
| Ex. | 0.136, | 4.4 | 5.7 | 5.8 | 4.1 | 121 | 6050 | 675 | 14398 |
| 10-2 | 0.118 | | | | | | | | |

* All data @ 1000 nits, CE = current efficiency; CIEx and CIEy are the x and y color coordinates according to the C.I.E. chromaticity scale (Commission Internationale de L'Eclairage, 1931). RawT50 is the time in hours for a device to reach one-half the initial luminance at the lifetest luminance given. Projected T50 is the projected lifetime at 1000 nits using an accelerator factor of 1.7.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. An aryl-substituted anthracene compound having at least one D.

2. The compound of claim 1, which is at least 10% deuterated.

3. The compound of claim 1, which is at least 50% deuterated.

4. The compound of claim 1, which is 100% deuterated.

5. The compound of claim 1, said compound having Formula I: 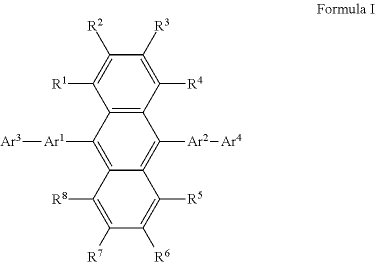
wherein:
R¹ through R⁸ are the same or different at each occurrence and are selected from the group consisting of H, D, alkyl, alkoxy, aryl, aryloxy, diarylamino, siloxane, and silyl;
Ar¹ and Ar² are the same or different and are selected from the group consisting of aryl groups; and
Ar³ and Ar⁴ are the same or different and are selected from the group consisting of H, D, and aryl groups;
wherein the compound has at least one D.

6. The compound of claim 5, wherein the at least one D is on a substituent group on an aryl ring.

7. The compound of claim 5, wherein at least one of R¹ through R⁸ is D.

8. The compound of claim 5, wherein R¹ through R⁸ are selected from H and D.

9. The compound of claim 5, wherein at least one of R¹ through R⁸ is selected from alkyl, alkoxy, aryl, aryloxy, diarylamino, siloxane, and silyl, and the remainder of R¹ through R⁸ are selected from H and D.

10. The compound of claim 9, wherein R² is selected from alkyl and aryl.

11. The compound of claim 5, wherein at least one of Ar¹ through Ar⁴ is a deuterated aryl.

12. The compound of claim 5, wherein Ar³ and Ar⁴ are selected from D and deuterated aryls.

13. The compound of claim 5, wherein Ar¹ through Ar⁴ are at least 20% deuterated.

14. The compound of claim 5, wherein Ar¹ and Ar² are selected from the group consisting of phenyl, naphthyl, phenanthryl, and anthracenyl.

15. The compound of claim 5, wherein Ar³ and Ar⁴ are selected from the group consisting of phenyl, naphthyl, phenanthryl, anthracenyl, phenylnaphthylene, naphthylphenylene, and a group having Formula II: 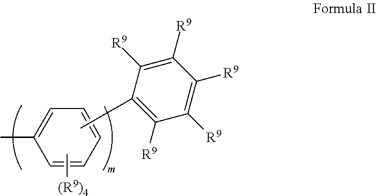
where:
R⁹ is the same or different at each occurrence and is selected from the group consisting of H, D, alkyl, alkoxy, diarylamino, siloxane and silyl, or adjacent R⁹ groups may be joined together to form an aromatic ring; and
m is the same or different at each occurrence and is an integer from 1 to 6.

16. The compound of claim 5, wherein Ar³ and Ar⁴ are selected from the group consisting of phenyl, naphthyl, phenylnaphthylene, naphthylphenylene, and a group having Formula III: 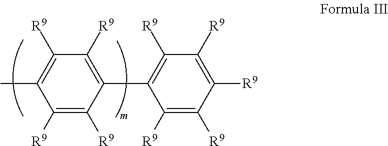
where:
R⁹ is the same or different at each occurrence and is selected from the group consisting of H, D, alkyl, alkoxy, siloxane and silyl, or adjacent R⁹ groups may be joined together to form an aromatic ring; and
m is the same or different at each occurrence and is an integer from 1 to 6.

17. The compound of claim 5, wherein at least one of Ar¹ through Ar⁴ is a heteroaryl group.

18. The compound of claim 17, wherein the heteroaryl group is selected from carbazole, benzofuran, and dibenzofuran.

19. The compound of claim 5, wherein at least one of R¹ through R⁸ is D and at least one of Ar¹ through Ar⁴ is a deuterated aryl.

20. A compound selected from Intermediate A through Intermediate M and H1 through H13.

21. An organic electronic device comprising a first electrical contact layer, a second electrical contact layer, and at least one active layer therebetween, wherein the active layer comprises an aryl-substituted anthracene compound having at least one D.

22. The device of claim 21, wherein the aryl-substituted anthracene compound has Formula I: 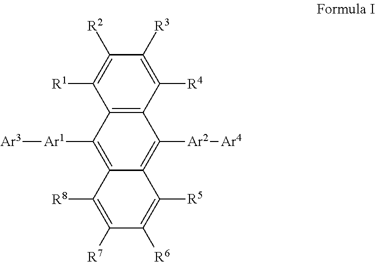
wherein:
R¹ through R⁸ are the same or different at each occurrence and are selected from the group consisting of H, D, alkyl, alkoxy, aryl, aryloxy, diarylamino, siloxane, and silyl;
Ar¹ and Ar² are the same or different and are selected from the group consisting of aryl groups; and
Ar³ and Ar⁴ are the same or different and are selected from the group consisting of H, D, and aryl groups;
wherein the compound has at least one D.

23. The device of claim 22, wherein the active layer consists essentially of the compound having Formula I and one or more electroluminescent compounds.

24. The device of claim 23, wherein the electroluminescent compound is selected from the group consisting of amino-substituted chrysenes and amino-substituted anthracenes.

25. The device of claim 23, further comprising a buffer layer between the first electrical contact layer and the active layer.

26. The device of claim 25, wherein the buffer layer comprises at least one electrically conductive polymer and at least one fluorinated acid polymer.
